(19) United States
(12) Patent Application Publication
(10) Pub. No.: US 2026/0202424 A1
(43) Pub. Date: Jul. 16, 2026

(54) PROTEIN BIOMARKERS FOR PROGNOSIS OF LIVER FIBROSIS

(71) Applicant: NEDERLANDSE ORGANISATIE VOOR TOEGEPAST-NATUURWETENSCHAPPELIJK ONDERZOEK TNO, s-GRAVENHAGE (NL)

(72) Inventors: Jan Roeland Occo HANEMAAIJER, Voorhout (NL); Arentje VAN KOPPEN, Bilthoven (NL); Lars VERSCHUREN, De Meem (NL)

(21) Appl. No.: 19/135,623

(22) PCT Filed: Dec. 7, 2022
(86) PCT No.: PCT/NL2022/050706

§ 371 Date: Jun. 4, 2025

(51) Int. Cl. G01N 33/68

(52) U.S. Cl. CPC G01N 33/6893; G01N 2333/705; G01N 2800/085; G01N 2800/50; G01N 2800/52; G01N 2800/56; G01N 2800/60

(57) ABSTRACT

The invention relates to methods for typing and analysing blood, serum or plasma samples of subjects for the presence of protein biomarkers for prognosis of hepatic fibrosis, and to methods for classifying and prognosticating subjects on the basis of such protein biomarkers.

### FIELD OF THE INVENTION

The invention relates to the field of non-alcoholic fatty liver disease (NAFLD) and liver fibrosis. More specifically, the invention relates to methods and means for prognosis of NAFLD and hepatic fibrosis, based on protein biomarkers in blood, serum or plasma.

### BACKGROUND OF THE INVENTION

Non-alcoholic fatty liver disease (NAFLD) is a spectrum of chronic liver diseases, encompassing fatty liver, non-alcoholic steatohepatitis (NASH) and hepatic fibrosis. NAFLD affects 25% of the global adult population and is the most common chronic liver disease worldwide. Non-alcoholic steatohepatitis (NASH) is the advanced form of NAFLD, a chronic disease characterized by excessive fat accumulation in the liver, hepatic necroinflammation and fibrosis progression. NASH can progress to cirrhosis and subsequent hepatocellular carcinoma.

No therapies have been approved for NAFLD or NASH till date, although many clinical trials have been initiated. However, all of these have failed, many in late stages. Several reasons for failure to trials in cirrhotic patients have been suggested (Ratziu et al. 2020), including: 1) patient selection, especially the divergence between the target of a drug and the stage of the disease at which it is administered as early and late stages may not be driven by the same mechanisms or have the same therapeutic targets; and 2) insufficient diagnostic and prognostic tools for classifying patients by disease stage and progression.

Liver biopsy is currently the standard in NAFLD diagnosis and prognosis. However, biopsy is an expensive and invasive procedure with a risk of complications, such as bleedings with associated morbidity and mortality, and shows variability in sampling. In addition, several non-invasive methods (such as MRI) are being developed to measure the elasticity of the liver, which is a measure of fibrosis (the more fibrosis, the lower tissue elasticity). But these methods are laborious, expensive, and not sensitive, and only patients with severe fibrosis can be identified. In addition, some blood-based markers exist, but they also are not sensitive, cannot discriminate between the various stages of disease, and are only suitable for diagnosis of late stage NASH or cirrhosis, but not for early stages and prognostics.

For example, tests based on plasma cytokeratin 18 (CK18) fragment levels (Cusi et al., 2014), soluble macrophage activation marker CD163 (Kazankov et al., 2016), a panel includes age, sex, AST, BMI, AST/ALT ratio, and serum hyaluronic acid (Palekar et al., 2006) have been described, but only with limited or late stage diagnostic value. Loomba et al. (2019) describe a serum test measuring alpha2 macroglobulin (A2M), hyaluronic acid (HA), and TIMP metallopeptidase inhibitor 1 (TIMP1).

Van Koppen et al. (2017) describe a gene expression signature for onset of NASH-related fibrosis in a NASH mouse model. Van Koppen et al. (2017) describe a gene expression signature for onset of NASH-related fibrosis in a NASH mouse model.

The Enhanced Liver Fibrosis (ELF) score is a marker set consisting of tissue inhibitor of metalloproteinases 1 (TIMP-1), amino-terminal propeptide of type III procollagen (PIIINP) and hyaluronic acid (HA) that is used to diagnose fibrosis in patients with NAFLD (Vali et al. J Hepatol. 2020 August; 73(2):252-262. doi: 10.1016/j.jhep.2020.03.036; Sharma et al. J Gastroenterol Hepatol. 2021 July; 36(7):1788-1802. doi: 10.1111/jgh.15482.).

Current methods and biomarkers are inadequate for predicting a risk of developing hepatic fibrosis or predicting the future severity of hepatic fibrosis. Therefore in practice often a combination of methods is used, however with limited success. There is a great need for prognostic blood-based biomarkers, preferably related to the active disease process.

With an increasing number of patients developing NASH-related end-stage liver disease (more than 3 million in the USA only) and pharmacological treatments on the horizon, there is a pressing need to develop biomarkers for prognostication of hepatic fibrosis, in particular in patients suffering from NAFLD and NASH, as this is key to successful prevention or and limitation of disease progression.

### SUMMARY OF THE INVENTION

It is an object of the present invention to provide biomarkers, in particular novel and improved biomarkers, for prognosis of hepatic fibrosis and progression thereof. In particular, it is an object of the invention to provide specific, sensitive biomarkers for non-invasive prognosis of hepatic fibrosis and progression thereof, in particular in the context of NAFLD and NASH.

The invention therefore provides a method for classifying a subject for being at risk of suffering from hepatic fibrosis or for being at risk of hepatic fibrosis progression, the method comprising determining the protein level of Scavenger Receptor Cysteine Rich Family Member With 5 Domains (SSC5D), Fibrillin 1 (FBN1), Thrombospondin 1 (THBS1), Urokinase-type plasminogen activator (uPA) and/or A disintegrin and metalloproteinase with thrombospondin motifs 2 (ADAMTS2) in a blood, serum or plasma sample of said subject and classifying the subject on the basis of said protein level.

In a further aspect, the invention provides a method for predicting a risk of development or progression of hepatic fibrosis in a subject, the method comprising determining the protein level of Scavenger Receptor Cysteine Rich Family Member With 5 Domains (SSC5D) Fibrillin 1 (FBN1), Thrombospondin 1 (THBS1), Urokinase-type plasminogen activator (uPA) and/or A disintegrin and metalloproteinase with thrombospondin motifs 2 (ADAMTS2) in a blood, serum or plasma sample of said subject and predicting risk of development or progression of hepatic fibrosis on the basis of said protein level.

In a further aspect, the invention provides a method for prognosticating severity of hepatic fibrosis in a subject, the method comprising determining the protein level of Scavenger Receptor Cysteine Rich Family Member With 5 Domains (SSC5D), Fibrillin 1 (FBN1), Thrombospondin 1 (THBS1), Urokinase-type plasminogen activator (uPA) and/or A disintegrin and metalloproteinase with thrombospondin motifs 2 (ADAMTS2) in a blood, serum or plasma sample of said subject and prognosticating severity of hepatic fibrosis on the basis of said protein level.

In a further aspect, the invention provides a method for determining whether a subject is suffering from an active hepatic fibrosis process, comprising determining the protein level of Scavenger Receptor Cysteine Rich Family Member With 5 Domains (SSC5D), Fibrillin 1 (FBN1), Thrombospondin 1 (THBS1), Urokinase-type plasminogen activator (uPA) and/or A disintegrin and metalloproteinase with thrombospondin motifs 2 (ADAMTS2) in a blood, serum or plasma sample of said subject and determining the presence of an active fibrosis process on the basis of said protein level.

In a further aspect, the invention provides a method for analysing a blood, serum or plasma sample of a subject, the method comprising determining the protein level of Scavenger Receptor Cysteine Rich Family Member With 5 Domains (SSC5D), Fibrillin 1 (FBN1), Thrombospondin 1 (THBS1), Urokinase-type plasminogen activator (uPA) and/or A disintegrin and metalloproteinase with thrombospondin motifs 2 (ADAMTS2) in said sample.

In a further aspect, the invention provides a method for typing a blood, serum or plasma sample from subject, the method comprising determining the protein level of Scavenger Receptor Cysteine Rich Family Member With 5 Domains (SSC5D), Fibrillin 1 (FBN1), Thrombospondin 1 (THBS1), Urokinase-type plasminogen activator (uPA) and/or A disintegrin and metalloproteinase with thrombospondin motifs 2 (ADAMTS2) in a blood, serum or plasma sample of said subject and typing said sample on the basis of said protein level.

In a further aspect, the invention provides a method for classifying and treating a subject, the method comprising:
determining the protein level of Scavenger Receptor Cysteine Rich Family Member With 5 Domains (SSC5D), Fibrillin 1 (FBN1), Thrombospondin 1 (THBS1), Urokinase-type plasminogen activator (uPA) and/or A disintegrin and metalloproteinase with thrombospondin motifs 2 (ADAMTS2) in a blood, serum or plasma sample of said subject,
classifying the subject as being at risk of suffering from hepatic fibrosis, for being at risk of hepatic fibrosis progression or as suffering from progressive hepatic fibrosis based on said protein level,
providing treatment to the subject classified as being at risk of suffering from hepatic fibrosis.

In a further aspect, the invention provides a method for assigning subjects to a clinical trial for treatment or prevention of hepatic fibrosis or non-alcoholic steatohepatitis (NASH) associated with hepatic fibrosis, the method comprising classifying subjects as being at risk of suffering from hepatic fibrosis or for being at risk of hepatic fibrosis progression and/or prognosticating severity of hepatic fibrosis with a method according to the invention and assigning subjects that are classified and/or prognosticated to said clinical trial.

In a preferred embodiment, a method of the invention comprises determining the protein level of SSC5D, FBN1 and/or THBS1 in said blood, serum or plasma sample.

In a preferred embodiment, a method of the invention comprises determining the protein level of SSC5D in said blood, serum or plasma sample.

In a preferred embodiment, a method of the invention comprises determining the protein levels of at least two proteins selected from SSC5D, FBN1, THBS1, uPA and ADAMTS2 in said blood, serum or plasma sample.

In a preferred embodiment, a method of the invention comprises determining the protein levels of SSC5D and FBN1 in said blood, serum or plasma sample.

In a preferred embodiment, a method of the invention comprises determining the protein levels of SSC5D, FBN1 and THBS1 in said blood, serum or plasma sample.

In a preferred embodiment, a method of the invention comprises determining the protein levels of SSC5D and THBS1 in said blood, serum or plasma sample.

In a preferred embodiment, a method of the invention comprises determining the protein levels of FBN1 and THBS1 in said blood, serum or plasma sample.

In a preferred embodiment, a method of the invention comprises determining the protein levels of SSC5D and uPA in said blood, serum or plasma sample.

In a preferred embodiment, a method of the invention comprises determining the protein levels of SSC5D and ADAMTS2 in said blood, serum or plasma sample.

In a preferred embodiment, a method of the invention comprises determining the protein levels of SSC5D, uPA, ADAMTS2 and FBN1 in said blood, serum or plasma sample.

In a preferred embodiment, a method of the invention comprises determining the protein levels of SSC5D, ADAMTS2 and FBN1 in said blood, serum or plasma sample.

In a preferred embodiment, a method of the invention comprises determining the protein levels of SSC5D, uPA and ADAMTS2 in said blood, serum or plasma sample.

In a preferred embodiment, a method of the invention comprises determining the protein levels of SSC5D, uPA and FBN1 in said blood, serum or plasma sample.

The hepatic fibrosis can be any type of hepatic fibrosis. However, in preferred embodiments, the hepatic fibrosis is hepatic fibrosis associated with NALFD, in particular NASH. As used herein “associated with NAFLD” or “associated with NASH” means that the risk of suffering from or developing hepatic fibrosis is the risk of suffering from of developing NAFLD or NASH, respectively, with hepatic fibrosis.

In a preferred embodiment, a method of the invention comprises determining the protein level or levels of the protein biomarkers or combinations of protein markers that are listed in FIGS. 1 and 3.

In a preferred embodiment, in a method of the invention in addition to the determined protein level or levels of the biomarkers disclosed herein, other parameters are taken into account in the method of the invention, in particular included in the value for the protein levels. Examples of such parameters include the age, gender, body-mass-index (BMI), ELF score, Alanine aminotransferase (ALT) level, aspartate aminotransferase (AST) level, hemoglobin A1C level, C-reactive protein level, plasma triglyceride level, plasma cholesterol level, MRE (magnetic resonance elastography) and genetic predisposition, e.g. presence of SNP's, to hepatic fibrosis and/or NAFLD of the subject. Said parameters are for instance included in the value for the determined protein levels. In one preferred embodiment, the ELF score is further determined and taken into account in a method in accordance with the invention, and/or taken into account in the value for the determined protein level or levels.

The subject from which the sample is taken can be any subject, i.e. not yet suffering from hepatic fibrosis or already suffering from hepatic fibrosis. In a preferred embodiment, the subject from which the sample is taken is suffering from fatty liver or fatty liver disease, in particular from non-alcoholic fatty liver disease (NAFLD), or NASH without hepatic fibrosis, or suspected of suffering therefrom. In other preferred embodiments, the subject is suffering from hepatic fibrosis. In further preferred embodiment the subject from which the sample is taken is having an ELF score of below 9.5.

In a preferred embodiment, in a method of the invention said subject is not diagnosed as suffering from hepatic fibrosis at the time of sampling.

In a preferred embodiment, in a method of the invention said subject has been diagnosed as not suffering from hepatic fibrosis at the time of sampling.

In a further aspect, the invention provides a kit of parts comprising means for determining protein level of Scavenger Receptor Cysteine Rich Family Member With 5 Domains (SSC5D) and one or more of Urokinase-type plasminogen activator (uPA), Fibrillin 1 (FBN1), A disintegrin and metalloproteinase with thrombospondin motifs 2 (ADAMTS2) and Thrombospondin 1 (THBS1), wherein said means comprises binding molecules specific for SSC5D and one or more of uPA, FBN1, ADAMTS2 and THBS1.

In preferred embodiment, a kit of parts of the invention comprises means as defined herein for determining the protein level or levels of the protein biomarkers or combinations of protein markers that are listed in FIGS. 1 and 3.

### DETAILED DESCRIPTION

As used herein, “to comprise” and its conjugations is used in its non-limiting sense to mean that items following the word are included, but items not specifically mentioned are not excluded. In addition the verb “to consist” may be replaced by “to consist essentially of” meaning that a compound or adjunct compound as defined herein may comprise additional component(s) than the ones specifically identified, said additional component(s) not altering the unique characteristic of the invention.

The articles “a” and “an” are used herein to refer to one or to more than one (i.e., to at least one) of the grammatical object of the article. By way of example, “an element” means one element or more than one element.

The word “approximately” or “about” when used in association with a numerical value (e.g. approximately 10, about 10) preferably means that the value may be the given value (e.g. 10), plus or minus 5% of the value (e.g. 10, plus or minus 5%), preferably plus or minus 1% of the value.

The use of the alternative (e.g., “or”) should be understood to mean either one, both, or any combination thereof of the alternatives.

The terms “treat” or “treatment” refer to inhibiting the disease or disorder, i.e., halting or reducing its development or at least one clinical symptom of the disease or disorder, and/or to relieving symptoms of the disease or condition. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

“Non-alcoholic fatty liver disease” or “NAFLD” refers to a spectrum of chronic liver diseases, encompassing fatty liver or non-alcoholic fatty liver (NAFL), to non-alcoholic steatohepatitis (NASH) with inflammation and different degrees of fibrosis to cirrhosis. NAFLD is also referred to as metabolic-associated fatty liver disease (MAFLD).

“Fatty liver”, “Non-alcoholic fatty liver” or “NAFL” as used herein refers to early stage NAFLD, characterized by fatty liver, but an absence of inflammation and fibrosis. This stage is also referred to as steatosis. It refers to a condition characterized by excess fat buildup in the liver, but an absence of inflammation and fibrosis.

“Fibrosis” as used herein refers to the formation of fibrous tissue in response to e.g. inflammation and is characterized by myofibroblast differentiation and deposition of matrix protein, including collagen.

“Hepatic fibrosis” as used herein refers to fibrosis present and/or occurring in the liver. “Severity of hepatic fibrosis” refers to the degree of fibrosis. The severity can for instance be classified as moderate or severe fibrosis. The degree or severity of hepatic fibrosis, in particular in NAFLD and NASH, can further be expressed in fibrosis stages, for example fibrosis stages F0, F1, F2, F3 and F4, whereby a higher number indicates a more advanced fibrosis stage and a lower number indicates a less advanced fibrosis stage. In particular, F0 indicates NASH with inflammation but no fibrosis; F1 perisinusoidal or periportal fibrosis; F2 indicates perisinusoidal and portal/periportal fibrosis; F3 indicates bridging fibrosis (bridging perisinusoidal and portal/periportal fibrotic tissue); and F4 indicates cirrhosis (According to NASH CRB scoring system, as described in Kleiner et al. Hepatology 2005, 41(6): 1313-1321). Hence, a distinction can be made between no (e.g. F0), moderate (F1-F2) and severe (F3-F4) fibrosis. Hence, in some embodiments, “no fibrosis” refers to fibrosis stage F0, “moderate hepatic fibrosis” refers to fibrosis stages F1 and F2, and “severe hepatic fibrosis” refers to fibrosis stages F3 and F4. However, a skilled person understand that classification of no, moderate and severe fibrosis can also be made without reference to this F-classification system.

As used herein “progressive hepatic fibrosis” and “active hepatic fibrosis” refer to progression of the fibrotic process, i.e. an increase in fibrous tissue in the liver over time. I.e. it generally refers to an increase in severity of hepatic fibrosis. In preferred embodiments, “progressive hepatic fibrosis” means that hepatic fibrosis is progressing to a more advanced fibrosis severity or stage, meaning that the amount of fibrous tissue is increased. E.g. no fibrosis is progressing to mild or moderate fibrosis, moderate fibrosis is progressing to severe fibrosis, severe fibrosis is progressing such that fibrous tissue is increased, F0 is progressing to any of F1-F4, F1 is progressing to any of F2-F4, F2 is progressing to F3 or F4, or F3 is progressing to F4.

“Non-alcoholic steatohepatitis” or “NASH” refers to NAFLD wherein fat accumulation is associated with varying degrees of inflammation (hepatitis) and varying degrees of fibrosis in the liver.

“Cirrhosis” as used herein refers to a late stage of hepatic fibrosis where the liver is characterized by high accumulation of matrix proteins and loss of liver functionality.

The term “analysing” as used herein refers to determining protein levels of indicated protein(s) in a blood, serum or plasma sample of a subject. Preferably, analysing comprises quantifying the protein levels, either absolutely or relative to a reference value.

The term “typing” as used herein refers In preferred embodiments, it comprises predicting the risk of hepatic fibrosis and/or predicting severity of hepatic fibrosis. Predicting severity of hepatic fibrosis preferably refers to differentiating the risk at moderate and severe hepatic fibrosis, more preferably differentiating the risk at no, moderate and severe hepatic fibrosis. Methods of typing in accordance with the invention are in particular suitable for prognosis of a subject suffering from fatty liver from which the sample is derived.

The term “prognosis” and “prognosticating” as used herein is defined as a prediction of a probable outcome, in particular for the prediction of future development or presence or future progression of hepatic fibrosis, i.e. at a later timepoint.

The term “prediction” of “predicting” is used herein to refer to the likelihood that a subject will develop or suffer from hepatic fibrosis or particular severity of hepatic fibrosis in the future, in particular within 6 years.

The term “at risk of” refers to the risk of an event that may occur in the future. For instance, “at risk of suffering from hepatic fibrosis” means the risk of suffering from hepatic fibrosis in the future, and “at risk of hepatic fibrosis progression” means the risk that the hepatic fibrosis will progress in the future. Determining a risk of progression of hepatic fibrosis refers to determining the likelihood that an active fibrosis process is currently present, because the presence of an active fibrosis process means that the hepatic fibrosis is likely to progress within time.

The present inventors have identified a set of protein biomarkers that are useful in prognosis of hepatic fibrosis, in particular in prognosis of hepatic fibrosis progression. In addition, the biomarkers are suitable for predicting the severity of such fibrosis, i.e. the risk of suffering from no hepatic fibrosis, moderate hepatic fibrosis or severe hepatic fibrosis. I.e. the biomarkers are useful in determining whether a subject that is currently not suffering from hepatic fibrosis is likely to do so in the future, and whether a subject that is already suffering from fibrosis is likely to suffer from more severe hepatic fibrosis in the future. The biomarkers are suitable for prognosis of any hepatic fibrosis. However, in preferred embodiments, the hepatic fibrosis is hepatic fibrosis associated with NAFLD or NASH. The biomarkers makes it possible in a minimal invasive way to provide such prognosis without the need for liver biopsy, making it possible to a) improve monitoring strategy of subjects at risk of development or progression of hepatic fibrosis, b) treat only subjects who need pharmaceutical intervention, and c) to stratify patient groups for clinical trials based on their prognosis. Until now all clinical trials for new NASH/fibrosis drugs have failed, partly due to the great variability in patients.

The biomarkers were identified in a unique way, using a mix of transcriptomics, dynamic proteomics, translational animal model for fibrosis, patient transcriptomics data, patient blood serum data, and selection of suitable biomarker assays. The starting point was not to perform a phishing expedition (as often used for biomarkers), but use a novel approach of combining gene expression with the formation of new collagen, which is a hallmark of progressive hepatic fibrosis, in a translational animal model. By basing the biomarkers on newly formed collagen, biomarkers of progressive hepatic fibrosis could be identified.

The upregulated genes were further analysed, and a final set of biomarkers was selected based on relevance in human samples and differential protein expression in blood samples. These biomarkers were validated in patient blood samples and using statistical tools, methods and algorithms were defined for a single biomarker and a selected number of biomarkers which can be used as a prognostic blood-based test to predict the risk of developing or progressing of hepatic fibrosis, and the severity thereof, based on the prediction of the presence of progressive fibrosis.

In a first aspect, the invention provides a method for analysing a blood, serum or plasma sample of a subject, the method comprising determining the protein level of Scavenger Receptor Cysteine Rich Family Member With 5 Domains (SSC5D), Fibrillin 1 (FBN1), Thrombospondin 1 (THBS1), Urokinase-type plasminogen activator (uPA) and/or A disintegrin and metalloproteinase with thrombospondin motifs 2 (ADAMTS2) in said sample. Also provided is a method for determining the protein level of Scavenger Receptor Cysteine Rich Family Member With 5 Domains (SSC5D), Fibrillin 1 (FBN1), Thrombospondin 1 (THBS1), Urokinase-type plasminogen activator (uPA) and/or A disintegrin and metalloproteinase with thrombospondin motifs 2 (ADAMTS2) in said sample. Preferably, protein levels are quantified. Provided is therefore a method of quantifying protein level in a blood, serum or plasma sample comprising determining the protein level of Scavenger Receptor Cysteine Rich Family Member With 5 Domains (SSC5D), Fibrillin 1 (FBN1), Thrombospondin 1 (THBS1), Urokinase-type plasminogen activator (uPA) and/or A disintegrin and metalloproteinase with thrombospondin motifs 2 (ADAMTS2) in the sample. Also provided is a method for typing a blood, serum or plasma sample from subject, the method comprising determining the protein level of Scavenger Receptor Cysteine Rich Family Member With 5 Domains (SSC5D), Fibrillin 1 (FBN1), Thrombospondin 1 (THBS1), Urokinase-type plasminogen activator (uPA) and/or A disintegrin and metalloproteinase with thrombospondin motifs 2 (ADAMTS2) in a blood, serum or plasma sample of said subject and typing said sample on the basis of said protein level. Preferably, protein levels are quantified. In a preferred embodiment, the protein level of SSC5D is determined and optionally quantified. In a further preferred embodiment, the protein levels of one or more of uPA, FBN1, ADAMTS2 and THBS1 in the sample are further determined and optionally quantified. In a further preferred embodiment, the protein levels of one the combinations of protein as listed in FIGS. 1 and 3 having an Area under the Curve (AUROC) higher than 0.55, preferably higher than 0.60, more preferably higher than 0.65, are determined and preferably quantified.

A value for the protein levels of said protein or proteins is preferably compared with a value for the protein level of the same protein or protein levels of the same proteins, respectively, in a reference. In one embodiment, protein level or levels are preferably compared with the protein level of the same protein or protein levels of the same proteins, respectively, in a reference. The subject from which the sample is taken can be any subject, i.e. not yet suffering from hepatic fibrosis or already suffering from hepatic fibrosis. In a preferred embodiment, the subject from which the sample is taken is suffering from fatty liver or fatty liver disease, in particular from non-alcoholic fatty liver disease (NAFLD), or NASH without hepatic fibrosis, or suspected of suffering therefrom. In other preferred embodiments, the subject is suffering from hepatic fibrosis. In further preferred embodiment the subject form which the sample is taken is having an ELF score of below 9.5.

In a preferred embodiment, the protein levels of SSC5D, FBN1 and/or THBS1 are determined and the subject is classified on the basis thereof and/or the risk of developing hepatic fibrosis is determined on the basis thereof.

In a preferred embodiment, the protein level of SSC5D is are determined and the subject is classified on the basis thereof and/or the risk of developing hepatic fibrosis is determined on the basis thereof.

The biomarkers of the present invention are suitable for prognosis of hepatic fibrosis, more specifically for predicting the risk of developing hepatic fibrosis, for predicting the risk of progression of already present hepatic fibrosis and for predicting the severity of future hepatic fibrosis. As such the biomarkers are suitable for detecting the presence of active or progressive hepatic fibrosis. In one aspect, the invention therefore provides a method for classifying a subject for being at risk of suffering from hepatic fibrosis or for being at risk of hepatic fibrosis progression, the method comprising determining the protein level of SSC5D, FBN1, THBS1, uPA and/or ADAMTS2 in a blood, serum or plasma sample of said subject and classifying the subject on the basis of said protein level. Classifying a subject for being at risk of suffering from hepatic fibrosis in particular is of subjects not yet suffering from hepatic fibrosis. Classifying a subject for being at risk of hepatic fibrosis progression is in particular of subject suffering from hepatic fibrosis. In another aspect, the invention provides a method for predicting risk of development or progression of hepatic fibrosis in a subject, the method comprising determining the protein level of SSC5D, FBN1, THBS1, uPA and ADAMTS2 in a blood, serum or plasma sample of said subject and predicting risk of development or progression of hepatic fibrosis on the basis of said protein level. Also provided is a method for determining whether a subject is suffering from an active hepatic fibrosis process, comprising determining the protein level of SSC5D, FBN1, THBS1, uPA and ADAMTS2 in a blood, serum or plasma sample of said subject and determining the presence of an active fibrosis process on the basis of said protein level.

The hepatic fibrosis can be any type of hepatic fibrosis. However, in preferred embodiments, the hepatic fibrosis is hepatic fibrosis associated with NALFD, in particular NASH. As used herein “associated with NAFLD” or “associated with NASH” means that the risk of suffering from or developing hepatic fibrosis is the risk of suffering from of developing NAFLD or NASH, respectively, with hepatic fibrosis.

In a preferred embodiment, the protein levels of SSC5D, FBN1 and/or THBS1 are determined and the subject is classified on the basis thereof and/or the risk of developing hepatic fibrosis is determined on the basis thereof.

In a preferred embodiment, the protein level of SSC5D is are determined and the subject is classified on the basis thereof and/or the risk of developing hepatic fibrosis is determined on the basis thereof.

In a further preferred embodiment, the protein levels of SSC5D or one the combinations of protein as listed in FIGS. 1 and 3 having an AUROC higher than 0.55, preferably higher than 0.60, more preferably higher than 0.65, are determined.

The biomarkers of the present invention are also suitable for predicting the severity of hepatic fibrosis. In particular, it can be predicted whether the subject is at risk of suffering from moderate or severe hepatic fibrosis, or from no, moderate or severe hepatic fibrosis. In one embodiment, severity of hepatic fibrosis is predicted after the risk of developing hepatic fibrosis is predicted. However, the risk of development or progression of hepatic fibrosis and the severity thereof can also be predicted in a single analysis.

Hence, in one preferred embodiment, classifying a subject for being at risk of suffering from hepatic fibrosis or for being at risk of hepatic fibrosis progression comprises predicting the severity of hepatic fibrosis. Preferably, the method comprises classifying a subject for being at risk of suffering from no, moderate or severe hepatic fibrosis. Similarly, in one preferred embodiment, predicting a risk of development or progression of hepatic fibrosis in a subject comprises predicting the severity of hepatic fibrosis. Preferably, the method comprises predicting the development of moderate or severe hepatic fibrosis. In some embodiments, the severity of hepatic fibrosis, in particular of moderate or severe hepatic fibrosis, is determined if the subject is classified as being at risk of suffering from hepatic fibrosis, or at risk of hepatic fibrosis progression or if it is predicted that the subject is at risk of development or progression of hepatic fibrosis.

In one aspect the invention provides a method for predicting the severity of hepatic fibrosis in a subject, the method comprising determining the protein level of Scavenger Receptor Cysteine Rich Family Member With 5 Domains (SSC5D), Fibrillin 1 (FBN1), Thrombospondin 1 (THBS1), Urokinase-type plasminogen activator (uPA) and/or A disintegrin and metalloproteinase with thrombospondin motifs 2 (ADAMTS2) in a blood, serum or plasma sample of said subject and predicting the severity of hepatic fibrosis on the basis of said protein level. In preferred embodiments, predicting the severity of hepatic fibrosis comprises predicting a risk of developing moderate or severe hepatic fibrosis or predicting the risk of developing no, moderate or severe fibrosis. In some embodiments, predicting the severity of hepatic fibrosis comprises predicting a risk of developing F1, F2, F3, F4, F1/F2 or F3/F4 stage hepatic fibrosis associated with NAFLD or NASH.

In preferred embodiments, a method of the invention, in particular for classifying a subject for being at risk of suffering from hepatic fibrosis or for predicting a risk of developing hepatic fibrosis, comprises determining the protein level of SSC5D in the sample and preferably classifying the subject on the basis of said protein level or predicting risk of developing hepatic fibrosis on the basis of said protein level.

In preferred embodiments, a method of the invention, in particular for classifying a subject for being at risk of suffering from hepatic fibrosis, for classifying a subject for being at risk of hepatic fibrosis progression or for predicting a risk of development or progression of hepatic fibrosis, comprises determining the protein level of SSC5D, and optionally one or more of FBN1, THBS1, uPA and ADAMTS2, in the sample and preferably classifying the subject on the basis of said protein level or predicting risk of developing hepatic fibrosis on the basis of said protein level.

In preferred embodiments, a method of the invention, in particular for classifying a subject for being at risk of suffering from hepatic fibrosis, for classifying a subject for being at risk of hepatic fibrosis progression or for predicting a risk of development or progression of hepatic fibrosis, comprises determining the protein level of FBN1 in the sample and preferably classifying the subject on the basis of said protein level or predicting risk of developing hepatic fibrosis on the basis of said protein level.

In preferred embodiments, a method of the invention, in particular for classifying a subject for being at risk of suffering from hepatic fibrosis, for classifying a subject for being at risk of hepatic fibrosis progression or for predicting a risk of development or progression of hepatic fibrosis, comprises determining the protein level of FBN1, and optionally one or more of SSC5D, THBS1, uPA and ADAMTS2, in the sample and preferably classifying the subject on the basis of said protein level or predicting risk of developing hepatic fibrosis on the basis of said protein level.

In preferred embodiments, a method of the invention, in particular for classifying a subject for being at risk of suffering from hepatic fibrosis, for classifying a subject for being at risk of hepatic fibrosis progression or for predicting a risk of development or progression of hepatic fibrosis, comprises determining the protein level of THBS1, and optionally one or more of SSC5D, FBN1, uPA and ADAMTS2, in the sample and preferably classifying the subject on the basis of said protein level or predicting risk of developing hepatic fibrosis on the basis of said protein level.

In preferred embodiments, a method of the invention, in particular for classifying a subject for being at risk of suffering from hepatic fibrosis, for classifying a subject for being at risk of hepatic fibrosis progression or for predicting a risk of development or progression of hepatic fibrosis, comprises determining the protein level of THBS1 in the sample and preferably classifying the subject on the basis of said protein level or predicting risk of developing hepatic fibrosis on the basis of said protein level.

In preferred embodiments, such method of the invention comprises determining the protein level of SSC5D and FBN1 in the sample and preferably classifying the subject on the basis of said protein level or predicting risk of developing hepatic fibrosis on the basis of said protein level.

In preferred embodiments, such method of the invention comprises determining the protein level of SSC5D, FBN1 and THBS1 in the sample and preferably classifying the subject on the basis of said protein level or predicting risk of developing hepatic fibrosis on the basis of said protein level.

In preferred embodiments, such method of the invention comprises determining the protein level of SSC5D and THBS1 in the sample and preferably classifying the subject on the basis of said protein level or predicting risk of developing hepatic fibrosis on the basis of said protein level.

In preferred embodiments, such method of the invention comprises determining the protein level of FBN1 and THBS1 in the sample and preferably classifying the subject on the basis of said protein level or predicting risk of developing hepatic fibrosis on the basis of said protein level.

In further preferred embodiments, a method of the invention, in particular methods comprising predicting the severity of hepatic fibrosis as defined herein, comprises determining the protein level of SSC5D, and optionally FBN1, THBS1, uPA and/or ADAMTS2, in the sample and preferably classifying the subject on the basis of said protein level or predicting risk and severity of future hepatic fibrosis on the basis of said protein level.

In further preferred embodiments, a method of the invention, in particular methods comprising predicting the severity of hepatic fibrosis as defined herein, comprises determining the protein level of SSC5D in the sample and preferably classifying the subject on the basis of said protein level or predicting risk and severity of future hepatic fibrosis on the basis of said protein level.

In further preferred embodiments, a method of the invention, in particular methods comprising predicting the severity of hepatic fibrosis as defined herein, comprises determining the protein level of FBN1, and optionally SSC5D, THBS1, uPA and/or ADAMTS2, in the sample and preferably classifying the subject on the basis of said protein level or predicting risk and severity of future hepatic fibrosis on the basis of said protein level.

In further preferred embodiments, a method of the invention, in particular methods comprising predicting the severity of hepatic fibrosis as defined herein, comprises determining the protein level of FBN1 in the sample and preferably classifying the subject on the basis of said protein level or predicting risk and severity of future hepatic fibrosis on the basis of said protein level.

In further preferred embodiments, a method of the invention, in particular methods comprising predicting the severity of hepatic fibrosis as defined herein, comprises determining the protein level of uPA, and optionally SSC5D, FBN1, THBS1, and/or ADAMTS2, in the sample and preferably classifying the subject on the basis of said protein level or predicting risk and severity of future hepatic fibrosis on the basis of said protein level.

In further preferred embodiments, a method of the invention, in particular methods comprising predicting the severity of hepatic fibrosis as defined herein, comprises determining the protein level of uPA in the sample and preferably classifying the subject on the basis of said protein level or predicting risk and severity of future hepatic fibrosis on the basis of said protein level.

In further preferred embodiments, a method of the invention, in particular methods comprising predicting the severity of hepatic fibrosis as defined herein, comprises determining the protein level of ADAMTS2, and optionally SSC5D, FBN1, THBS1 and/or uPA, in the sample and preferably classifying the subject on the basis of said protein level or predicting risk and severity of future hepatic fibrosis on the basis of said protein level.

In further preferred embodiments, a method of the invention, in particular methods comprising predicting the severity of hepatic fibrosis as defined herein, comprises determining the protein level of ADAMTS2 in the sample and preferably classifying the subject on the basis of said protein level or predicting risk and severity of future hepatic fibrosis on the basis of said protein level.

In preferred embodiments, such method of the invention comprises determining the protein level of SSC5D and FBN1 in the sample and preferably classifying the subject on the basis of said protein level or predicting risk and severity of future hepatic fibrosis on the basis of said protein level.

In preferred embodiments, such method of the invention comprises determining the protein level of SSC5D and uPA in the sample and preferably classifying the subject on the basis of said protein level or predicting risk and severity of future hepatic fibrosis on the basis of said protein level.

In preferred embodiments, such method of the invention comprises determining the protein level of SSC5D and ADAMTS2 in the sample and preferably classifying the subject on the basis of said protein level or predicting risk and severity of future hepatic fibrosis on the basis of said protein level.

In preferred embodiments, such method of the invention comprises determining the protein level of SSC5D, uPA, ADAMTS2 and FBN1 in the sample and preferably classifying the subject on the basis of said protein level or predicting risk and severity of future hepatic fibrosis on the basis of said protein level.

In preferred embodiments, such method of the invention comprises determining the protein level of SSC5D, uPA and ADAMTS2 in the sample and preferably classifying the subject on the basis of said protein level or predicting risk and severity of future hepatic fibrosis on the basis of said protein level.

In preferred embodiments, such method of the invention comprises determining the protein level of SSC5D, uPA and FBN1 in the sample and preferably classifying the subject on the basis of said protein level or predicting risk and severity of future hepatic fibrosis on the basis of said protein level.

In preferred embodiments, such method of the invention comprises determining the protein level of SSC5D, ADAMTS2 and FBN1 in the sample and preferably classifying the subject on the basis of said protein level or predicting risk and severity of future hepatic fibrosis on the basis of said protein level.

In preferred embodiments, such method of the invention comprises determining the protein level of uPA and ADAMTS2 in the sample and preferably classifying the subject on the basis of said protein level or predicting risk and severity of future hepatic fibrosis on the basis of said protein level.

In preferred embodiments, such method of the invention comprises determining the protein level of uPA, ADAMTS2 and FBN1 in the sample and preferably classifying the subject on the basis of said protein level or predicting risk and severity of future hepatic fibrosis on the basis of said protein level.

In preferred embodiments, such method of the invention comprises determining the protein level of uPA and FBN1 in the sample and preferably classifying the subject on the basis of said protein level or predicting risk and severity of future hepatic fibrosis on the basis of said protein level.

In preferred embodiments, such method of the invention comprises determining the protein level of ADAMTS2 and FBN1 in the sample and preferably classifying the subject on the basis of said protein level or predicting risk and severity of future hepatic fibrosis on the basis of said protein level.

In some embodiments, a method of the invention comprises determining the protein level or protein levels of the protein biomarkers or combination of protein biomarkers as defined herein and no further protein levels.

In embodiments, methods of the present invention involve prognosis of and/or predicting a risk of development or progression of hepatic fibrosis. Hence, in embodiments, the methods are for predicting future development or progression of hepatic fibrosis. In other embodiments, the methods of the present invention involve prognosis of and/or predicting severity of hepatic fibrosis. In preferred embodiments, the methods are for predicting future severity of hepatic fibrosis. “Risk” in the context of the present invention relates to the probability that an event, i.e. hepatic fibrosis or progression thereof, or a specific severity of hepatic fibrosis, will occur over a specific time period. In preferred embodiments the prognosis or prediction with respect to the risk of a subject suffering from hepatic fibrosis is the prognosis or prediction that the subject will suffer from hepatic fibrosis within about 10 years, more preferably within about 8 years, more preferably within about 7 years, more preferably within about 6 years, e.g. in 2-6 years, in 3-6 years, in 4-6 years, in 5-6 years or in 6 years. Similarly, in preferred embodiments prognosis or prediction with respect to progression of hepatic fibrosis is the progression of hepatic fibrosis within about 10 years, more preferably within about 8 years, more preferably within about 7 years, more preferably within about 6 years, e.g. in 2-6 years, in 3-6 years, in 4-6 years, in 5-6 years or in 6 years. Similarly, in preferred embodiments the prognosis or prediction with respect to the severity of hepatic fibrosis that a subject will be suffering is the prognosis or prediction that the subject will suffer from the specific severity of hepatic fibrosis within about 10 years, more preferably within about 8 years, more preferably within about 7 years, more preferably within about 6 years, e.g. in 2-6 years, in 3-6 years, in 4-6 years, in 5-6 years or in 6 years.

A used herein a “subject” is preferably a human.

Classification and prediction in accordance with the present invention are suitable for subjects independent of the medical history of the subject. As shown in the Examples herein, the biomarkers proved successful in predicting hepatic fibrosis in subjects that were randomly included in a population screening. The subject can be any subject, i.e. not yet suffering from hepatic fibrosis or already suffering from hepatic fibrosis. Hence, in some embodiments, the subject is a healthy subject. As used herein “healthy subject” refers to an individual not known to suffer from hepatic fibrosis, preferably not known to suffer from NAFLD. In some embodiments, the subject is suspected of being at risk suffering from hepatic fibrosis, in particular hepatic fibrosis associated with NALFD, in particular NASH without hepatic fibrosis. In some embodiments, the subject is suffering from fatty liver, non-alcoholic fatty liver (NAFL), or NASH without hepatic fibrosis, i.e. suffering from fatty liver without present fibrosis. In some embodiments, the subject is suffering from hepatic fibrosis. In some preferred embodiment the subject has an ELF score of below 9.5.

In some preferred embodiments, the subject is not suffering from hepatic fibrosis at the time the blood, serum or plasma sample is obtained from the subject, also referred to as the time of sampling. In some embodiments, the subject is a subject who is not diagnosed as suffering from hepatic fibrosis at the time of sampling, in particular not diagnosed by a medical professional as suffering from hepatic fibrosis. In some embodiments, the subject is a subject that has been diagnosed as not suffering from hepatic fibrosis at the time of sampling, in particular diagnosed by a medical professional as not suffering from hepatic fibrosis. In some embodiments, the subject is a subject who is diagnosed as suffering from hepatic fibrosis, preferably having an ELF score of below 9.5.

Whether or not a subject is not suffering from hepatic fibrosis can be established by several methods known in the art, for instance but not necessarily by a medical professional. Similarly, whether or not a subject is suffering from fatty liver/NALFD without hepatic fibrosis can be established by several methods known in the art, for instance but not necessarily by a medical professional. E.g. fatty liver can be established by one of the following procedures or a combination thereof: blood analysis of in particular liver enzymes and/or proteins, analysis of ultrasound or computed tomography (CT) scan and analysis of liver biopsy. Liver stiffness measurement (LSM) methods can be used to detect hepatic fibrosis accompanying fatty liver and severity thereof, such as FibroScan®. Such methods measure stiffness of the liver using ultrasound, whereby an absence of fibrosis is characterized by an absent or low stiffness and the presence of fibrosis is characterized by high stiffness of the liver. Further, stiffness of the liver increases with increased severity of hepatic fibrosis. Another method to measure hepatic fibrosis is the Enhanced Liver Fibrosis (ELF) score, which is a marker set consisting of tissue inhibitor of metalloproteinases 1 (TIMP-1), amino-terminal propeptide of type III procollagen (PIIINP) and hyaluronic acid (HA). The ELF score is also used to distinguish different fibrosis stages, i.e. to determine severity of hepatic fibrosis. In preferred embodiments, a subject is considered as suffering from severe hepatic fibrosis if the ELF score is higher than about 9.5. In preferred embodiments, a subject is considered as not suffering from hepatic fibrosis or suffering from mild or moderate hepatic fibrosis if the ELF score is below about 9.5.

Preferably, in a method of the invention, the protein level or levels of SSC5D, FBN1, THBS1, uPA and/or ADAMTS2 or a value for said protein level or levels is compared with a reference value. It is further preferred that the subject is classified or the subjects risk is predicted based on said comparison. I.e. classification and risk prediction on the basis of determined protein level or levels preferably comprises comparing said protein level or levels or a value from said protein level or for said protein levels with a reference. More preferably, classifying and risk prediction are preferably performed based on a comparison of the protein level or levels or a value for said protein level or levels in blood, serum or plasma of the subject that is classified or the subjects risk is predicted in comparison with a reference value. Hence, in a preferred embodiment, a method of classifying a subject in accordance with the invention comprises comparing the protein level or levels of SSC5D, FBN1, THBS1, uPA and/or ADAMTS2 in a blood, serum or plasma sample of said subject with a reference value. In one embodiment, a value for said protein level or levels of SSC5D, FBN1, THBS1, uPA and/or ADAMTS2 is compared with a reference value.

The term “reference” or “reference sample” as used herein refers to a value or sample, in particular a blood, serum or plasma sample, that comprises proteins, from a subject or subjects not suffering from hepatic fibrosis, or from a subject or subjects who is/are known to suffer from hepatic fibrosis, in particular hepatic fibrosis associated with NAFLD or NASH. If the reference is of a subject or subjects who is/are known to suffer from hepatic fibrosis, the severity of the hepatic fibrosis is preferably also known, in particular moderate or severe fibrosis or e.g. stages F1, F2, F3 and F4 or stages F0, F1-F2 and F3-F4. The term “reference value” refers to a value determined for a reference sample. In preferred embodiments, the reference value is a value for a reference sample from a subject or subjects as defined herein.

The reference sample is preferably the same type of sample as the sample of the subject to be classified. The reference value is preferably a value determined in the same type of sample of the subject to be classified. I.e. if protein level or levels are determined in a blood sample, the reference is preferably also a blood sample; if protein level or levels are determined in a serum sample, the reference is preferably also a serum sample; if protein level or levels are determined in a plasma sample, the reference is preferably also a plasma sample. In a preferred embodiment, both the sample of the subject (i.e. the test sample) and the reference sample are a serum sample.

The reference can be a reference sample obtained from a single subject, and/or the reference value can be the value of the particular protein level in a sample of a single subject. It is, however, preferred, that the reference value is the average of the particular protein level or levels or a value for said protein level or levels in a plurality of subjects, i.e. a plurality of subjects not suffering from hepatic fibrosis, or a plurality of subjects known to suffer from hepatic fibrosis, preferably a plurality of subjects suffering from fatty liver, in particular NAFL or NASH without hepatic fibrosis. Said plurality is for instance at least 5 subjects, at least 10 subjects, at least 20 subjects, at least 30 subjects, at least 50 subjects, at least 75 subjects, or at least 100 subjects.

Based on a comparison with the protein level or levels of SSC5D, FBN1, THBS1, uPA and/or ADAMTS2 in the reference, it can be determined whether a subject is at risk of suffering from hepatic fibrosis, or is at risk from a particular hepatic fibrosis severity and/or fibrosis stage.

For instance, it can be determined whether the protein level or levels or a value for said protein level or levels in the sample is higher than or similar to a reference value. Whether or not protein level or levels or a value for said protein level or levels is higher than or similar to a reference value can be determined using statistical methods that are appropriate and well-known in the art, generally with a probability value of less than five percent chance of the change being due to random variation. It is well within the ability of a skilled person to determine the amount of increase or similarity that is considered significant. Preferably, “higher than” is at least 10, at least 20, at least 40, or at least 50% higher than the reference value. Preferably, “similar to” is at most 20% difference, more preferably at most 10% difference between protein level determined and the reference value.

In preferred embodiments, a subject is classified as being at risk of suffering from hepatic fibrosis as defined herein, if the protein level or levels of SSC5D, FBN1, THBS1, uPA and/or ADAMTS2 in the sample of the subject are higher than the protein level or levels of SSC5D, FBN1, THBS1, uPA and/or ADAMTS2 in a reference sample from a subject or subjects not suffering from hepatic fibrosis. This is in particular the case if the protein level of a single protein, e.g. SSC5D, FBN1, THBS1, uPA or ADAMTS2, preferably SSC5D, is determined. Preferably, “higher than” is at least 10, at least 20, at least 40, or at least 50% higher than the reference value.

In other preferred embodiments, a subject is classified as being at risk of suffering from hepatic fibrosis as defined herein, if the protein level or levels of SSC5D, FBN1, THBS1, uPA and/or ADAMTS2 in the sample of the subject is similar to the protein level or levels of SSC5D, FBN1, THBS1, uPA and/or ADAMTS2 in a reference sample from a subject or subjects known to suffer from hepatic fibrosis. This is in particular the case if the protein level of a single protein, e.g. SSC5D, FBN1, THBS1, uPA or ADAMTS2, preferably SSC5D, is determined. Preferably, “similar to” is at most 20% difference, more preferably at most 10% difference between protein level determined and the reference value.

In further preferred embodiments, a subject is classified as being at risk of suffering from a particular hepatic fibrosis severity as defined herein, in particular moderate or severe fibrosis, if the protein level or levels of SSC5D, FBN1, THBS1, uPA and/or ADAMTS2 in the sample of the subject are higher than the protein level or levels of SSC5D, FBN1, THBS1, uPA and/or ADAMTS2 in a reference sample from a subject or subjects not suffering from hepatic fibrosis or similar to the protein level or levels of SSC5D, FBN1, THBS1, uPA and/or ADAMTS2 in a reference sample from a subject or subjects known to suffer from the same hepatic fibrosis severity. This is in particular the case if the protein level of a single protein, e.g. SSC5D, FBN1, THBS1, uPA or ADAMTS2, preferably SSC5D, is determined. Preferably, “higher than” is at least 10, at least 20, at least 40, or at least 50% higher than the reference value.

As another example, the difference or similarity between the protein level or levels or a value for said level or levels in a sample and a reference protein level or levels or a reference value for said level or levels can be determined by determining a correlation or ratio of the protein level, levels or value in the sample and the reference protein level, levels or value, respectively. For example, it can be determined whether a value for the protein level or levels in a sample correlates to the value for the protein level or levels of the same proteins in a reference. Said correlations between the values for the protein level or levels in the subjects sample and the reference, can be used to produce an overall similarity score for the protein biomarker or biomarkers used. A similarity score is a measure of the average correlation of a value for the protein level or levels of a set of proteins in a sample from a subject and a reference value. This correlation can be numerically expressed, e.g. using a correlation coefficient. Several correlation coefficients can be used. Preferred methods are parametric methods which assume a normal distribution of the data. Said correlation or similarity score can for instance be, but does not need to be, a numerical value between +1, indicative of a high correlation between the value for the protein levels in a sample of said subject and said reference, and −1, which is indicative of an inverse correlation. A threshold can be used to differentiate between samples classified as from subjects at risk of suffering from hepatic fibrosis or a particular severity thereof, e.g. a particular fibrosis stage, or samples classified as from subjects not suffering from hepatic fibrosis or a particular severity thereof, e.g. a particular fibrosis stage. Said threshold is an arbitrary value that allows for discrimination between samples from subjects and that is dependent of the method used. If a similarity threshold value is employed, it is preferably set at a value at which an acceptable number of subjects with a positive classification for e.g. at risk of suffering from hepatic fibrosis or a particular severity thereof, e.g. a particular fibrosis stage, would score as false negatives, and an acceptable number of subjects with a negative classification for e.g. suffering from hepatic fibrosis or a particular severity thereof, e.g. a particular fibrosis stage, would score as false positives.

The specific reference or reference sample and threshold that is used in a method of the invention for classifying a subject, depends on the specific method, and a skilled person is well capable of identifying and using an appropriate reference samples and reference values. Suitable references samples and values are provided in more detail herein below. In preferred embodiments, the coefficient for the protein level or levels or a value therefor is higher than the coefficient for the reference level, levels or value, respectively, if the reference is from a subject or subjects not suffering from hepatic fibrosis. Preferably, “higher than” is at least 10, at least 20, at least 40, or at least 50% higher than the reference value. In other preferred embodiments, the coefficient for the protein level or levels or a value therefor is similar to the coefficient for the reference level, levels or value, respectively, if the reference is from a subject or subjects known to suffer from hepatic fibrosis. Preferably, “similar to” is at most 20% difference, more preferably at most 10% difference between protein level determined and the reference value.

The level or levels of the proteins in a reference and/or a value for this level or levels are preferably stored on a computer, or on computer-readable media, to be used in comparisons to the level of level data from a sample of subject that is typed, analyzed or classified in accordance with the present invention. The term “reference value” refers to the protein level or a value for said protein level of a particular protein or of particular proteins in a reference, in particular the protein levels or a value for said protein levels of SSC5D, FBN1, THBS1, uPA and/or ADAMTS2 and preferred combinations of proteins as detailed herein.

In preferred embodiments, a value for the protein level or protein levels of SSC5D, FBN1, THBS1, uPA and/or ADAMTS2 is compared with a reference value. In one preferred embodiment, the reference value is the protein level or levels or a value for said protein level or levels of the same protein or proteins that are determined in the sample of the subject to be classified in a blood, serum or plasma reference sample.

A value for the protein level or levels of multiple proteins as described herein is preferably a weighted value. Similarly, the reference value is preferably a weighted value for the same protein level or levels in the reference sample. A value, e.g. a weighted value, for the protein level or levels can be determined by a person skilled in the art using any suitable method or algorithm, including ensemble methods that use multiple learning algorithms to obtain a predictive, i.e. prognostic, value. These values are then based on the protein level or levels for the subject to be tested and the protein level or levels in the reference sample. Non-limiting examples of suitable methods are random forest classification, gradient boosting, artificial neural networks (NN), Adaptive Synthetic Sampling Approach for Imbalanced Learning (ADASYN) and kernel principal component analysis (KPCA). Hence, in preferred embodiments of a method of the invention, a predictive, i.e. prognostic, value based on the protein level or levels of SSC5D, FBN1, THBS1, uPA and/or ADAMTS2 is determined with a multiple learning algorithm. A skilled person is well capable of determining such predicting value using a multiple learning algorithm based on samples obtained from subjects known to suffer from or not suffer from hepatic fibrosis, in particular progressive or active hepatic fibrosis, and the severity thereof, in particular obtained over time in order to achieve a predictive or prognostic value.

In one embodiment, a random forest classifier is determined for the protein level or levels that are determined in accordance with the present invention.

In one embodiment, an ADASYN classifier is determined for the protein level or levels that are determined in accordance with the present invention.

A skilled person is well capable of determining values for protein level or levels in a sample and reference values and suitable threshold values for SSC5D, FBN1, THBS1, uPA and/or ADAMTS2 according to the invention with a method as described herein.

In some embodiments, in addition to determined protein level or levels, other parameters are taken into account in the weighted value. In one embodiment, the ELF score is further determined and taken into account in the weighted value. In one embodiment, one or more parameters selected from age, gender, body-mass-index (BMI), Alanine aminotransferase (ALT) level, aspartate aminotransferase (AST) level, hemoglobin A1C level, C-reactive protein level, plasma triglyceride level, plasma cholesterol level, MRE (magnetic resonance elastography) and genetic predisposition, e.g. presence of SNP's, to hepatic fibrosis and/or NAFLD of the subject are taken into account in the weighted value.

In another embodiment, the value, in particular the weighted value, is based solely on the determined protein level or levels, of SSC5D, FBN1, THBS1, uPA and/or ADAMTS2 and specific combinations or proteins as defined herein, preferably combinations of proteins as indicated in FIGS. 1 and 3.

In one preferred embodiment the value, preferably the weighted value, for the protein levels is a value for the protein level of SSC5D. In one preferred embodiment the value, preferably the weighted value, for the protein levels is a value for the protein levels of SSC5D and uPA. In one preferred embodiment the value, preferably the weighted value, for the protein levels is a value for the protein levels of SSC5D, FBN1 and/or THBS1. In one preferred embodiment the value, preferably the weighted value, for the protein levels is a value for the protein levels of SSC5D. In one preferred embodiment the value, preferably the weighted value, for the protein levels is a value for the protein levels of preferred combinations of markers as disclosed in FIGS. 1 and 3. In one preferred embodiment the value, preferably the weighted value, for the protein levels is a value for the protein levels of preferred combinations of markers as disclosed herein.

As described herein above, no therapies have been approved for NAFLD or NASH, or hepatic fibrosis till date, although many clinical trials have been initiated, but all of these have failed, many in late stages. One reason for this failure is that until now it has been difficult to accurately determine disease activity or severity, such as presence or risk of hepatic fibrosis or severity thereof/fibrosis stage, in patients and thus to classify patients in clinical trials by disease activity or severity, e.g. disease stage. Now that the present invention makes it possible to predict the risk of developing and progression of hepatic fibrosis, predict the severity of future hepatic fibrosis in healthy subjects, or fatty liver, NALF or NASH patients without or with present hepatic fibrosis, it has become possible to assign subjects or patients to clinical trials and classify subjects or patients therein, based on e.g. the risk or severity, e.g. fibrotic stage, of hepatic fibrosis. The invention therefore also provides a method for assigning subjects to a clinical trial for treatment or prevention of hepatic fibrosis and/or non-alcoholic steatohepatitis (NASH), the method comprising classifying subjects as being at risk of suffering from hepatic fibrosis or for being at risk of hepatic fibrosis progression and/or prognosticating severity of hepatic fibrosis with a method according to the invention and assigning subjects that are classified and/or prognosticated to said clinical trial, in particular subjects that are classified as being at risk of suffering from hepatic fibrosis, are suffering from progressive hepatic fibrosis or a particular hepatic fibrosis severity/stage. In one preferred embodiment, said method comprises determining protein level of SSC5D in the sample. In one preferred embodiment said method comprises determining protein levels of SSC5D and uPA.

In one preferred embodiment said method comprises determining protein level or levels of SSC5D, FBN1 and/or THBS1. In one preferred embodiment said method comprises determining protein level of preferred combinations of markers as disclosed herein, in particular combinations of markers as disclosed in FIGS. 1 and 3.

In a method of the invention, the protein level of SSC5D, FBN1, THBS1, uPA and/or ADAMTS2 is or are determined in a blood, serum or plasma sample. In some embodiments, methods disclosed herein therefore comprise obtaining or providing a blood, serum or plasma sample from the subject. A blood sample of a subject can be obtained by any standard method, for instance by venous extraction. In some embodiments, methods disclosed herein comprise isolating protein, preferably total protein, from the blood, serum or plasma sample.

In a method of the invention, the protein level of one or more of SSC5D, uPA, FBN1, ADAMTS2 and THBS1 is determined.

Scavenger Receptor Cysteine Rich Family Member With 5 Domains (SSC5D) is identified by UniProtKB reference number A1L4H1.

Urokinase-type plasminogen activator (uPA) is identified by UniProtKB reference number P00749.

Fibrillin 1 (FBN1) is identified by UniProtKB reference number P35555.

A disintegrin and metalloproteinase with thrombospondin motifs 2 (ADAMTS2) is identified by UniProtKB reference number 095450.

Thrombospondin 1 (THBS1) is identified by UniProtKB reference number P07996.

Methods for determining protein levels in a sample are well known in the art. A protein level in a blood, serum or plasma, preferably serum, may be determined by any assay known to a skilled person. Examples of such assays are polyacrylamide gel electrophoresis, including two dimensional gel electrophoresis, multidimensional protein identification technology, ELISA, bead-based immunoassays, immuno-PCR using, for example, Thunder-Link® antibody-oligonucleotide conjugation kit (Innova Biosciences. Cambridge UK), surface plasmon resonance, liquid chromatography—tandem mass spectrometry (LC-MS/MS), multiplex assay such as Luminex, meso scaled discovery (MSD) and matrix-assisted laser desorption/ionization time-of-flight mass spectrometry (MALDI-TOF). Further examples of suitable assays are chemo-luminescence assays, fluorescence assays, mass spectrometry, affinity chromatography, Western blotting, Northern blotting, histology and protein expression chips, probes. Preferred are multiplex systems that can measure expression levels from different proteins at the same time.

Mass spectrometry is a suitable means of determining a level of expression of a protein. A preferred method comprises liquid chromatography coupled to tandem mass spectrometry in positive electrospray ionization mode. The LC-MS/MS analysis may be performed, for example by using an I-Class UPLC system connected to a Xevo TQS mass spectrometer Waters (Manchester, UK), or an Q Exactive mass spectrometer (Thermo Fisher). A suitable multiplex system for determining a protein level is multiple reaction monitoring (MRM), which is a quantitative MS-based approach.

The protein level or levels are preferably detected and quantified using an immunochemical assay, preferably employing binding molecules such as antibodies that specifically bind to a ligand on said proteins. A protein is an antigen for an binding molecule that specifically reacts with said protein.

The binding molecules, preferably antibodies, are preferably coupled to a solid support such as a bead, monolithic material or a multi-well array. The binding molecules, preferably antibodies, may be coupled directly, or indirectly, for example by coupling of a second binding molecule that specifically recognizes the first binding molecule that binds to a protein. Indirect coupling may be accomplished, for example, by coupling of antibody-binding molecules such as protein A, protein G, or a mixture of protein A and G to beads, monolithic material or array. Direct coupling may be accomplished, for example, by cross-linking, covalently binding or physically adsorbing said binding molecule, preferably antibody, to the solid support.

A preferred method for determining a level of expression of a protein or multiple proteins includes Enzyme-Linked Immuno Sorbent Assay (ELISA) and Flow Cytometric ImmunoAssay (FCIA).

In a competition ELISA, known amounts of an antigen are immobilized to a surface. A sample comprising unknown amounts of said antigen is added, and the antigen is subsequently complexed with a binding molecule that is preferably conjugated, directly or indirectly, to a detectable label such as a colorimetric label, a fluorescent label, a radioactive label or a chemiluminescent label, or an enzyme. Following washing, detection of the binding molecule that is complexed to the immobilized antigen is accomplished by assessing the conjugated label or enzyme activity via incubation with a substrate to produce a measurable product. The amount of label or enzyme activity is inversely proportional to the amount of antigen in the sample.

A further preferred assay is a sandwich ELISA, in which a receptacle is coated with a first binding molecule that is specific to a protein, termed “capture binding molecule”, and detection of bound protein is accomplished with a second binding molecule, termed “detection binding molecule”. It is preferred that the capture and detection binding molecules do not interfere with each other and can bind simultaneously to said protein.

Said coating of a receptacle or bead, preferably the surface of a receptacle or bead, may be performed directly or indirectly. Indirect coating may be accomplished, for example, by using a biotin-labeled capture binding molecule that is attached to a linker molecule, for example a U-PLEX Linker (Meso-Scale Discovery, Rockville, USA). The employment of different linker molecules for different capture antibodies allows the generation of arrayed spots on a receptacle, each of which will bind to a specific protein. Said receptacle preferably is a multi-well plate, such as a 24 well plate, a 96 well plate, a 192 well plate, or a 384 well plate, in which each of the wells comprises arrayed spots, whereby each of the spots will bind to a specific protein.

Said second binding molecule is preferably directly or indirectly conjugated to a detectable label such as a colorimetric label, a fluorescent label a radioactive label, or a chemiluminescent label, or an enzyme. Detection of the amount of enzyme-conjugated binding molecule is preferably performed by incubation with a substrate to produce a measurable product. As an alternative, turbidimetric assays are preferred, especially for competition ELISAs.

Detectable labels are well known in the art. A detectable label may be a fluorescent, luminescent, chemiluminescent and/or electrochemiluminescent moiety which, when exposed to specific conditions, may be detected. For example, a fluorescent label may be exposed to radiation (i.e. light) at a specific wavelength and intensity to cause excitation of the fluorescent label, thereby enabling it to emit detectable fluorescence at a specific wavelength that may be detected.

Alternatively, the detectable label may be an enzyme which is capable of converting a (preferably undetectable) substrate into a detectable product that can be visualized and/or detected. Suitable enzymes include horseradish peroxidase, phosphatase, phosphatase/pyrophosphatase and luciferase.

Alternatively, the detectable label may be a radioactive label, which may be incorporated by methods known in the art.

Indirect labeling of a binding molecule may be accomplished, for example, through conjugation of a binding molecule with biotin and reacting biotin with labelled or enzyme-linked avidin or streptavidin.

As an alternative, carbon coated wells may be equipped with electrodes that produce chemical energy when subjected to an electrical charge, such as the Multi-Array® and Multi-Spot® 96-well plates of Meso-Scale Discovery. When combined with a SULFO-TAG® antibody, the chemical energy is transformed to emitted light which is measured using a high-resolution CCD camera.

The methods disclosed herein classify a subject as being at risk of suffering from hepatic fibrosis or progressive or prognosticate severity thereof. Preferably, the methods predict the likelihood that a subject will suffering from or not suffering from hepatic fibrosis, progressive hepatic fibrosis or a particular hepatic fibrosis severity, in particular no, moderate or severe fibrosis. Depending on the likelihood and/or prognosticated progression and/or severity of hepatic fibrosis, and potentially other factors, such as age, gender and BMI of the subject and other health conditions, a treatment strategy can be assigned to the subject.

In one aspect, the invention therefore provides a method for treating, preferably for classifying and treating, a subject, the method comprising:
determining the protein level of Scavenger Receptor Cysteine Rich Family Member With 5 Domains (SSC5D), Fibrillin 1 (FBN1), Thrombospondin 1 (THBS1), Urokinase-type plasminogen activator (uPA) and/or A disintegrin and metalloproteinase with thrombospondin motifs 2 (ADAMTS2) in a blood, serum or plasma sample of said subject,
classifying the subject as being at risk of suffering from hepatic fibrosis, for being at risk of hepatic fibrosis progression or as suffering from progressive hepatic fibrosis based on said protein level,
providing treatment to the subject classified as being at risk of suffering from hepatic fibrosis.

Classification is preferably performed with a method according to the invention as disclosed herein. Classification is further preferably predicting a risk of developing or progression of hepatic fibrosis in the subject, with a method according to the invention as disclosed herein. Alternatively, or additionally, classification is determining that the subject is suffering from progressive or active hepatic fibrosis with a method of the invention. Alternatively, or additionally, classification is prognosticating the severity of hepatic fibrosis in the subject, with a method according to the invention.

In preferred embodiment, the protein level of at least SSC5D is determined in said blood, serum or plasma sample, and the subject is classified on the basis thereof.

In preferred embodiments, a method of the invention comprising treatment comprises comparing said determined protein level or a value for said protein level with a reference value as defined herein, preferably wherein said reference value is the protein level or levels of the same protein or proteins or a value for said protein level or levels in a blood, serum or plasma sample from one or more subjects.

In particular, the invention provides a method for assigning treatment to a subject who is predicted to be at risk of suffering from hepatic fibrosis or who is predicted to be at risk of suffering from a particular hepatic fibrosis severity of stage with a method of the invention.

Also provided is a method for determining a treatment schedule for a subject, comprising determining, using a method according to the invention as disclosed herein, whether a subject is at risk of suffering from or developing hepatic fibrosis or suffering from active hepatic fibrosis, in particular hepatic fibrosis associated with NALFD, in particular NASH. If the determination is positive, it can be determined if and how the individual can be treated.

In some embodiments, in particular if the subject is classified as being at risk of suffering from hepatic fibrosis, suffering from progressive hepatic fibrosis or is prognosticated to be at risk of suffering from a particular hepatic fibrosis severity of stage, treatment comprises regular examination of the status and/or monitoring of the condition of the liver of the subject. Such regular examination refers preferably to periodic examining, e.g. annually, bi-annually or three-monthly, examining of the status and/or monitoring of the condition of the liver, including e.g. blood analysis of in particular liver enzymes and/or proteins, analysis of ultrasound or computed tomography (CT) scan and analysis of liver biopsy, e.g. liver stiffness measurement (LSM) by e.g. FibroScan®.

In some embodiments, treatment comprises examination of the condition of the liver by referral to the appropriate medical specialist after a positive test outcome with a marker or combination of markers of the invention.

Treatment in the context of a method of the invention may further include life style changes, treatment with vitamin E, metformin, pioglitazone, liraglutide, obeticholic acid, cenicriviroc, aramchol, Resmetirom, Semaglutide and other GLP receptor modulators, Tropifexor, Liraglutide, Nidufexor, Aldafermin, FGF21 mimick or analogs, growth hormone analogs, PPAR modulators, Firsocostat and other ACC modulators, Belapectin and other Galectin-3 inhibitors, EDP-305 and other bile acid receptor modulators, MSDC-0602K and other mTOT regulators, Selonsertib and other ASK1 inhibitors, Obeticholic acid, Aramchol, Cenicriviroc mesilate, Resmetirom, Selonsertib Hydrochloride, ALS-L-1023 (AngioLab), Aldafermin, Apararenone, BFKB-8488A Genentech), Belapectin, CC-90001 (Celgene), CRV-431 (Hepion Pharmaceuticals), Cenicriviroc/tropifexor, Cilofexor tromethamine, Cotadutide, EDP-305 (Enanta Pharmaceuticals), EYP-001 (Enyo Pharma), Efruxifermin, Elafibranor, Epeleuton, Ezetimibe, Firsocostat, GRI-0621 (Glyoregimmune), HHALPC (Promethera Biosciences), HM-15211 (Hanmi), Imm-124-E (Immuron), Icosabutate, LPCN-1144 (Lipocine), LYS-006 (Novartis), Lanifibranor, Leronlimab, Licogliflozin diprolinate, MET-409 (Metacrine), MSDC-0602K (Cirius Therapeutics), Metformin hydrochloride/sildenafil citrate/L-leucine, Naltrexone hydrochloride, Namodenoson, Nitazoxanide, Oramed insulin (Oramed), PF-05221304 (Pfizer), PF-06835919 (Pfizer), PF-06865571 (Pfizer), PXL-770 (Poxel), Peqbelfermin, Pemafibrate, Pioqlitazone hydrochloride, SGM-1019 (Second Genome), Semaglutide, Silymarin, TERN-101 (Terns), TVB-2640 (Sagimet Biosciences), Tesamorelin, Tipelukast, Tirzepatide, Tropifexor, Ursodeoxycholic acid berberine, VK-2809 (Viking Therapeutics), Vupanorsen, ARO-HSD (Arrowhead Pharmaceuticals), BI089-100 (89bio), FT-4101 (FORMA Therapeutics), NN-6177 (Novo Nordisk), ZSP-1601 (Guangdong Raynovent Biotech), 25-OH Cholesterol 3beta-sulfate, 5(R)-Deuteropioglitazone, AGN-242266 (Allergan), ALB-127158 (ConSynance Therapeutics), AZD-2693 (AstraZeneca), CB-4211 (CohBar), CER-209 (ABIONYX Pharma), CM-101 (ChemomAb), DWP-10292 (Daewoong), FM-101 (Future Medicine), Foralumab, GB-1211 (Galecto Biotech), HEC-96719 (HEC Pharma), HPG-1860 (Hepagene Therapeutics), IDL-2965 (Indalo Therapeutics), ION-224 (Tonis Pharmaceuticals), Miricorilant, NGM-395 (NGM Biopharmaceuticals), NN-9500 (Novo Nordisk), Nimacimab (Bird Rock Bio), PAT-409 (Blade Therapeutics), PBI-4547 (Liminal BioSciences), TERN-201 (Terns), Technetium Tc 99m tilmanocept, XW-003 (Hangzhou Sciwind Biosciences), XSP-0678 (Guangdong Raynovent Biotech), AXA-1125 (Axcella Health), AXA-1957 (Axcella Health), ALN-HSD (Alnylam Pharmaceuticals), ASC-41 (Ascletis), HPN-01 (Hepanova), AGN-242256 (Allergan), ALY-688 (Allysta Pharmaceuticals), ANG-3070 (Angion Biomedica), ASC-42 (Ascletis), Atrosab, Atrosimab, Bemitil, Bromantane, CJ-14199 (CH HealthCare), CRB-4001 (Corbus Pharmaceuticals), EDP-297 (Enanta Pharmaceuticals), GDD-3898 (Lipidio Pharma), Gel-B (Gelesis), Hu-6 (Rivus Pharmaceuticals), INB-03 (INmune Bio), JT-194 (Jecure Therapeutics), NGM-386 (NGM Biopharmaceuticals), NVP-018 (Abliva), Nicodicosapent, PRX-106 (Protalix Biotherapeutics), RLA-8 (Beijing Institute Pharmacology Toxicol), RLBN-1127 (Acquist Therapeutics), RPC-8844 (Celgene), RT-200 (Renova Therapeutics), RT-210 (Renova Therapeutics), S-723595 (Shionogi), SC-410 (Sancilio), SCO-116 (SCOHIA), SFA-001 (Sinew Pharma), SP-1373 (Altimmune), SRT-015 (Seal Rock Therapeutics), TERN-501 (Terns), URK-614 (ImmuPharma), YH-25724 (Yuhan), liver transplantation or a combination thereof.

Also provided is a kit of parts comprising means for determining protein level of Scavenger Receptor Cysteine Rich Family Member With 5 Domains (SSC5D) and one or more of Urokinase-type plasminogen activator (uPA), Fibrillin 1 (FBN1), A disintegrin and metalloproteinase with thrombospondin motifs 2 (ADAMTS2) and Thrombospondin 1 (THBS1), wherein said means comprises binding molecules specific for SSC5D and one or more of uPA, FBN1, ADAMTS2 and THBS1. Said kit is preferably for analysing a sample in accordance with the invention, for typing a sample in accordance with the invention, for classifying a subject in accordance with the invention and/or for predicting a risk of developing hepatic fibrosis in a subject in accordance with the invention. Said means are preferably suitable for determining protein level of SSC5D and one or more of FBN1, THBS1, uPA and ADAMTS2, in a blood, serum or plasma sample. Such kit may comprise one or more of the following components: a container for collecting blood, serum or plasma, a container filled with preservative and/or one or more test tubes and other materials such as buffers and enzymes for analysis, and instructions for use.

The kit of parts according to the invention may be selected from any suitable assay and data processing apparatus and equipment, e.g. as described herein above. Said means for determining protein level of SSC5D and one or more of FBN1, THBS1, uPA and ADAMTS2, preferably are means for an immunochemical assay. Said means for determining protein levels for instance comprise binding molecules, preferably antibodies or antigen binding parts thereof, that specifically bind to SSC5D and one or more of FBN1, THBS1, uPA and ADAMTS2, or to an epitope in said proteins. The binding molecules, preferably antibodies or antigen binding parts thereof, may be coupled directly, or indirectly, to a detectable label, such as a colorimetric label, a fluorescent label, a radioactive label or a chemiluminescent label, or an enzyme. Said detectable label is preferably a non-amino acid label, for instance a fluorescent label, a radioactive label or a chemiluminescent label, in particular a non-amino acid fluorescent, radioactive or chemiluminescent label. Indirect coupling to a label is for example by coupling of a second, labeled binding molecule, preferably antibody, that specifically recognizes the first binding molecule that binds to the at least two proteins, preferably comprising SSC5D and one or more of FBN1, THBS1, uPA and ADAMTS2, or epitope of said proteins.

In preferred embodiment, a kit of parts of the invention comprises means as defined herein for determining the protein level or levels of the protein biomarkers or combinations of protein markers that are listed in FIGS. 1 and 3.

Also provided is a kit of parts according to the invention for use in a method of analysing a sample in accordance with the invention, a method of typing a sample in accordance with the invention and/or a method for classifying a subject in accordance with the invention, and/or a method of prognosticating in accordance with the invention.

In one embodiment, in addition to the determined protein level or levels of the biomarkers disclosed herein, other parameters are taken into account in a method of classifying a subject of the invention, in particular included in the value for the protein levels. Examples of such parameters include the age, gender, body-mass-index (BMI), ELF score, Alanine aminotransferase (ALT) level, aspartate aminotransferase (AST) level, hemoglobin A1C level, C-reactive protein level, plasma triglyceride level, plasma cholesterol level, MRE (magnetic resonance elastography) and genetic predisposition, e.g. presence of SNP's, to hepatic fibrosis and/or NAFLD of the subject. Said parameters are for instance included in the value for the determined protein levels. In one preferred embodiment, the ELF score is further determined and taken into account in classification or prediction in accordance with the invention, and/or taken into account in the value for the determined protein level or levels.

Features may be described herein as part of the same or separate aspects or embodiments of the present invention for the purpose of clarity and a concise description. It will be appreciated by the skilled person that the scope of the invention may include embodiments having combinations of all or some of the features described herein as part of the same or separate embodiments.

The invention will be explained in more detail in the following, non-limiting examples.

### BRIEF DESCRIPTION OF THE DRAWINGS

FIG. 1: Random Forest results for each biomarker in predicting active hepatic fibrosis using AUROC (prediction) calculation. Sensitivity and Specificity of each of the combination was calculated.

FIG. 2: Example of AUROC for SSC5D in prognosticating hepatic fibrosis.

FIG. 3: Random Forest results for each biomarker in predicting no hepatic fibrosis, moderate hepatic fibrosis and severe hepatic fibrosis using AUROC (prediction) calculation. Sensitivity and Specificity of each of the combination was calculated.

### EXAMPLES

### 1. Methods.

### Human Material Collection—Identification of Potential Biomarkers

Medical archives of 2 University Medical Centers in the Netherlands were used to collect 817 Formalin Fixed Paraffin Embedded (FFPE) liver biopsies, related to NASH and fibrosis, and matched serum samples. Samples of which there was suspicion of non-NASH-related fibrosis, e.g. Hepatitis B (HBV), Hepatitis C (HCV), Alcoholic Steatohepatitis (ASH) and Auto-immune Hepatitis (AIH) were excluded. In total 155 samples were included and pathological re-examination was performed by an independent pathologist. Scoring was performed according the SAF (Steatosis, Activity and Fibrosis) algorithm (PMID: 22707395; DOI: 10.1002/hep.25889). Both FFPE biopsies and matched serum samples from NASH patients with different fibrosis stages were used to determine blood-based biomarkers for NASH and fibrosis.

### Human Material Collection—Prognostic Study

Medical archives of a University Medical Center in the Netherlands was used to collect sera from 80 individuals at two time points. The individuals were included in a random population screening. The interval between the two time points of sample collection was 6 years. At the second time point the presence of fibrosis was diagnosed using transient elastography (Fibroscan®) [The Fibroscan device (Echosens) works by measuring shear wave velocity. In this technique, a 50-MHz wave is passed into the liver from a small transducer on the end of an ultrasound probe. The probe also has a transducer on the end that can measure the velocity of the shear wave (in meters per second) as this wave passes through the liver. The shear wave velocity can then be converted into liver stiffness, which is expressed in kiloPascal. Essentially, the technology measures the velocity of the sound wave passing through the liver and then converts that measurement into a liver stiffness measurement]. At both time points the ELF test was applied to assess the presence or absence of fibrosis. The ELF™ (Enhanced Liver Fibrosis) Test from Siemens Healthineers (Erlangen, Germany) is a routine, standardized, direct-biomarker panel for assessment of fibrosis due to NASH. The ELF score combines three serum biomarkers, namely Hyaluronic acid (HA), Procollagen III N-terminal peptide (PIIINP) and Tissue inhibitors of metalloproteinase (TIMP-1).

### FFPE RNA Isolation and Sequencing

Total RNA was extracted from FFPE liver samples using glass beads and RNeasy FFPE kit (Qiagen). Special lysis and incubation conditions enabled reverse formaldehyde modifications of RNA. The lysis buffer efficiently released RNA from tissue sections while avoiding further RNA degradation. After removing ribosomal RNA, cDNA synthesis was performed (Qiagen). Thereafter, cDNA was ligated with the sequencing adapters and amplified by PCR. Quality and yield of the amplicon was measured (Fragment Analyzer, Agilent Technologies, Amstelveen, The Netherlands), DV200>40% was used as quality control for sufficient quality. Library prep was performed using NEBNext Ultra II Directional RNA Library Prep Kit for Illumina (NEB #E7760S/L). Clustering and DNA sequencing, using the Illumina NovaSeq6000, was performed according to manufacturer's protocols by service provider GenomeScan B.V (Leiden, the Netherlands), yielding 40-100 million sequencing clusters per sample and 2×150 bp Paired-End reads (PE) per cluster. The count data served as input for the statistical analysis using DEseq2 package (PMID: 25516281; doi:10.1186/s13059-014-0550-8). Selected differentially expressed genes (DEGs), corrected for multiple testing (available in DEseq2 package), were used for expression analyses.

### Steps Taken to Identify Potential Biomarkers

Various steps were taken to uncover the biomarkers and biomarker sets for of NASH/fibrosis and fibrogenesis. In a preclinical animal study genes and proteins were identified which correlated with fibrosis and which could be detected at a time before there was manifestation of the disease itself (PMID: 29276754; DOI: 10.1016/j.jcmgh.2017.10.001). These mouse signature genes were translated into the human homologues.

RNA sequencing data from FFPE liver samples from NASH/fibrosis patients were used in which from the mouse signature dataset those genes were selected which were increased at a transcriptional level in patients with NASH/fibrosis vs non-fibrotic patients. The selected set of genes was further evaluated by selection for gene protein products which were produced in the liver. For this, literature and the human protein atlas were used to evaluate what the protein products of the genes of interest were, whether these protein products were produced in the liver, whether they were proteins present in the circulation and whether they could be detected in human plasma or serum. Furthermore, it was evaluated if the proteins were linked to fibrosis. Note that proteins that were NOT linked to liver or fibrosis were NOT immediately excluded from the list as they might represent novel biomarkers. This resulted in a set of 11 potential circulating biomarker candidates (Insulin-like growth factor-binding protein 7 (IGFBP7), Thrombospondin 1 (THBS1), Tenacin C (TNC), C-X-C motif chemokine ligand 10 (CXCL10), Urokinase-type plasminogen activator (uPA), Annexin A3 (ANXA3), Semaphorin 4D (SEMA4D), Fibrillin 1 (FBN1), Versican (VCAN), A disintegrin and metalloproteinase with thrombospondin motifs 2 (ADAMTS2), Protein Associated with MYC (PAM) and Scavenger Receptor Cysteine Rich Family Member With 5 Domains (SSC5D)), which was used for further evaluating in clinical serum samples.

### Measuring Biomarkers in Sera Using ELISA

The various biomarkers were analyzed in sera using commercial ELISA's. The ELISA's were first validated by testing recovery and linearity of dilution. The ELISA's used were obtained from R&D Systems (Minneapolis, USA), Abbexa (Cambridge, UK), Cell Signalling Technology (Danvers, USA) and Millipore Sigma (St Louis, USA). The ELISA's were applied according the manufacturer's instructions and using dilutions in the linear range of the ELISA. For 12 potential biomarker candidates a suitable assay was identified.

### Using Algorithm to Predict Active Fibrosis and Fibrosis Severity

Identification of prognostic biomarkers to predict presence of fibrosis at 6-year follow-up was performed in two steps. First, the recursive feature elimination with random forest was applied for the biomarkers THBS1, PAM, VCAN, FBN1, CXCL10, ADAMTS2, IGFBP7, TNC, SEMA4D, uPA and SSCD5 to rank and identify the most important biomarkers. After that, a random forest was used to predict presence of fibrosis at 6-year follow-up based on varying biomarkers. Five (THBS1, FBN1, ADAMTS2, uPA and SSCD5) out of the 11 biomarkers showed to have prognostic potential to predict presence of fibrosis at 6-years follow up. Repeated 5-fold cross validation was applied to evaluate AUROC (Area Under the Receiver Operating Characteristics curve), Sensitivity and Specificity by using these biomarkers. Forest plots displaying the average and confidence intervals of model metrics were plotted to qualitatively select the best combination of biomarkers to predict presence of fibrosis.

The same approach was used to identify the severity of fibrosis (i.e., no fibrosis, moderate fibrosis, severe fibrosis) at 6-year follow-up. The ELF score at baseline was used to exclude those individuals that have fibrosis at baseline (ELF>9.5). The ELF score was included in the analyses with blood biomarkers as a benchmark and to determine whether ELF score in combination with the biomarkers can improve predictive value.

### 2. Results Prognostic Study

### A: Biomarkers Levels at Baseline and Fibrosis at 6 Years

Eleven biomarkers candidates (THBS1, PAM, VCAN, FBN1, CXCL10, ADAMTS2, IGFBP7, TNC, SEMA4D, uPA and SSCD5) were used for analysis in sera of individuals at baseline. ELF score was calculated at baseline and 6 years follow up. LSM (Liver Stiffness Measurement) was determined by Fibroscan at 6 years follow up. Individuals were subdivided in 2 groups at baseline based on LSM and ELF score at 6 years, whereby 40 individuals were identified as having no hepatic fibrosis at 6 years and 40 individuals were identified as having hepatic fibrosis at 6 years. Table 1 shows data for all biomarkers. For 2 of the biomarkers a significant increased level at baseline was observed in the group with fibrosis at 6 years follow up.

TABLE 1
Biomarker analysis in sera from individuals at t = 0 and
t = 6 years. THBS1 (μg/ml), PAM (ng/ml), VCAN (ng/ml),
FBN1 (ng/ml), CXCL10 (pg/ml), ADAMTS2 (ng/ml), IGFBP7 (ng/ml),
TNC (ng/ml), SEMA4D (μg/ml), uPA (pg/ml), SSCD5 (ng/ml),
Enhanced Liver Function (ELF) test (reference value), Liver
Stiffness Measurement (LSM, kPa). 1 indicates the average
concentration of the relevant biomarker at t = 0 in the
group of individuals that showed no fibrosis after 6 years
follow up. 2 indicates the average concentration of the relevant
biomarker at t = 0 in the group of individuals that showed
fibrosis after 6 years follow up.

| | Baseline (t = 0) biomarker concentration | | | | |
|---|---|---|---|---|---|
| | 1) No fibrosis | | 2) Fibrosis | | |
| | at 6 years | | at 6 years | | |
| | average | stdev | average | stdev | t-test |
| TNC (ng/ml) | 12.52 | 3.44 | 14.96 | 5.90 | 0.027 |
| FBN1 (ng/ml) | 35.82 | 20.93 | 36.27 | 33.10 | 0.942 |
| SEMA4D (μg/ml) | 0.56 | 0.17 | 0.60 | 0.21 | 0.304 |
| ADAMTS2 (ng/ml) | 27.82 | 12.80 | 29.66 | 24.21 | 0.674 |
| PAM (ng/ml) | 63.67 | 14.35 | 66.41 | 16.07 | 0.425 |
| SSC5D (ng/ml) | 2.15 | 1.30 | 4.29 | 3.68 | 0.001 |
| VCAN (ng/ml) | 101.33 | 13.57 | 101.17 | 13.75 | 0.960 |
| Urokinase | 783.16 | 198.53 | 775.43 | 171.13 | 0.852 |
| PLAU (pg/ml) | | | | | |
| THBS1 (μg/ml) | 26.89 | 7.96 | 26.94 | 13.49 | 0.985 |
| IGFBP7 (ng/ml) | 140.68 | 27.99 | 148.01 | 27.08 | 0.238 |
| CXCL10 (pg/ml) | 197.34 | 119.08 | 184.18 | 84.97 | 0.569 |
| ELF (ref. value) | 8.48 | 0.69 | 8.98 | 0.86 | 0.007 |
| Fibroscan | 4.45 | 1.09 | 9.90 | 3.51 | 0.000 |
| (LSM; 6 years) | | | | | |
| ELF (ref. value; | 9.03 | 0.81 | 9.57 | 0.98 | 0.009 |
| 6 years) | | | | | |

B: Data Analysis of Biomarkers for Absence or Presence of Fibrosis after 6 Years Follow Up (2 Groups)

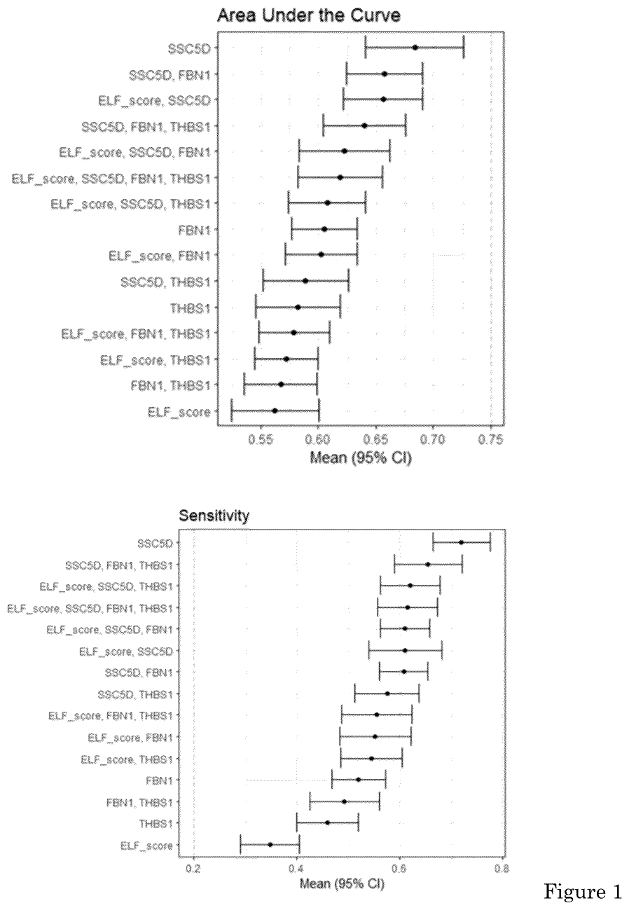

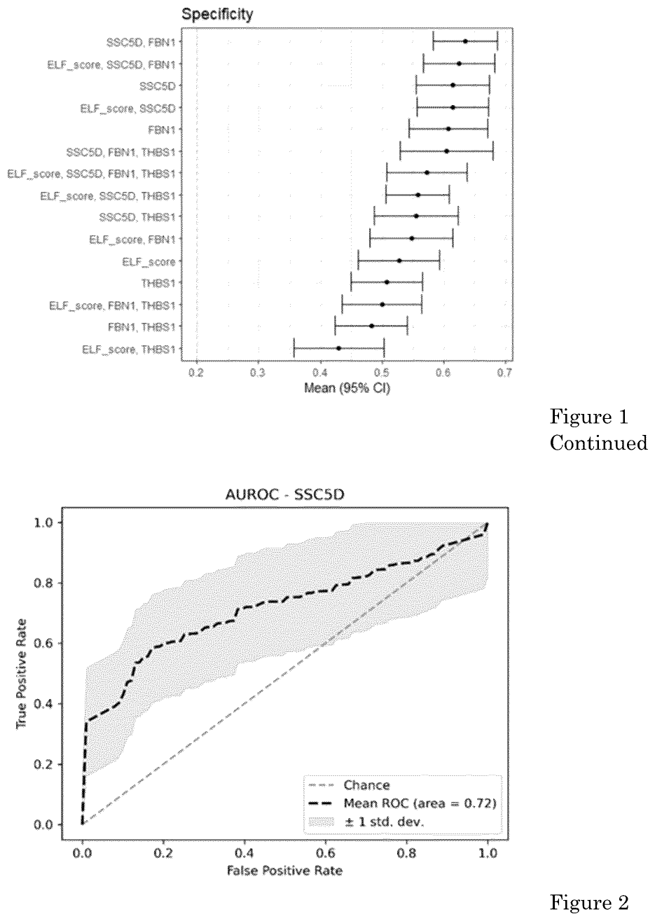

Next, using baseline serum data, Random Forest analysis showed that several biomarkers alone and in combination were able to predict the presence or absence of fibrosis after 6 years follow up (FIGS. 1 and 2). The best prediction was observed using SSC5D alone resulting in predictive value of 70% (AUROC=0.7). Predictive value for fibrosis at 6 years follow up by ELF score was 56% (AUROC=0.56), which was improved by combination with our biomarkers.

### C: Data Analysis of Biomarkers for No, Moderate or Severe Fibrosis (3 Groups)

Eleven biomarkers candidates (THBS1, PAM, VCAN, FBN1, CXCL10, ADAMTS2, IGFBP7, TNC, SEMA4D, uPA and SSCD5) were used for analysis in sera of individuals at baseline. ELF score was calculated at baseline and at 6 years follow up. LSM (Liver Stiffness Measurement) was determined by Fibroscan at 6 years follow up. Individuals were subdivided into 3 groups (no fibrosis, moderate fibrosis and severe fibrosis) based on LSM and ELF score at 6 years follow up.

Table 2 shows data for all biomarkers. Table 2 shows the biomarkers with a significant increased level at baseline in the groups with moderate or severe fibrosis at 6 years follow up as compared to no fibrosis.

TABLE 2
Biomarker analysis in sera from NASH/fibrosis individuals at t = 0
and t = 6 years. THBS1 (μg/ml), PAM (ng/ml), VCAN (ng/ml), FBN1
(ng/ml), CXCL10 (pg/ml), ADAMTS2 (ng/ml), IGFBP7 (ng/ml), TNC (ng/ml),
SEMA4D (μg/ml), uPA (pg/ml), SSCD5 (ng/ml), Enhanced Liver Function
(ELF) test (reference value), Liver Stiffness Measurement (LSM, kPa).

| | Baseline (t = 0) biomarker concentration | | | | | | | |
|---|---|---|---|---|---|---|---|---|
| | | | 2) Moderate | | | 3) Severe | | |
| | 1) no fibrosis | | fibrosis | | | fibrosis | | |
| | average | stdev | average | stdev | t-test | average | stdev | t-test |
| TNC (ng/ml) | 12.26 | 4.03 | 15.18 | 6.69 | 0.101 | 14.74 | 5.12 | 0.027 |
| FBN1 (ng/ml) | 31.98 | 16.14 | 31.38 | 14.97 | 0.902 | 41.42 | 44.90 | 0.942 |
| SEMA4D (μg/ml) | 0.59 | 0.20 | 0.61 | 0.21 | 0.819 | 0.59 | 0.22 | 0.304 |
| ADAMTS2 (ng/ml) | 27.17 | 10.91 | 27.34 | 11.45 | 0.960 | 32.09 | 32.92 | 0.674 |
| PAM (ng/ml) | 69.86 | 12.94 | 62.49 | 15.36 | 0.106 | 70.52 | 16.15 | 0.425 |
| SSC5D (ng/ml) | 2.06 | 1.34 | 3.70 | 2.40 | 0.011 | 4.94 | 4.70 | 0.001 |
| VCAN (ng/ml) | 101.27 | 11.92 | 100.14 | 14.01 | 0.783 | 102.26 | 13.75 | 0.960 |
| Urokinase PLAU (pg/ml) | 767.29 | 153.23 | 836.12 | 181.60 | 0.198 | 711.71 | 136.37 | 0.852 |
| THBS1 (μg/ml) | 27.15 | 6.32 | 27.86 | 18.09 | 0.869 | 25.96 | 6.02 | 0.985 |
| IGFBP7 (ng/ml) | 142.53 | 28.93 | 146.75 | 24.77 | 0.618 | 149.34 | 29.90 | 0.238 |
| CXCL10 (pg/ml) | 213.87 | 159.21 | 199.66 | 103.37 | 0.735 | 167.93 | 58.38 | 0.569 |
| ELF (ref. value) | 8.44 | 0.66 | 9.19 | 0.89 | 0.004 | 8.75 | 0.80 | 0.000 |
| Fibroscan (LSM; 6 years) | 3.63 | 0.61 | 7.70 | 0.49 | 0.000 | 12.21 | 3.84 | 0.007 |
| ELF (ref. value; 6 years) | 8.83 | 0.63 | 9.56 | 0.84 | 0.004 | 9.59 | 1.12 | 0.009 |

1) indicates the average concentration of the relevant biomarker at t = 0 in the group of individuals that showed no fibrosis after 6 years follow up.
2) indicates the average concentration of the relevant biomarker at t = 0 in the group of individuals that showed moderate fibrosis after 6 years follow up.
3) indicates the average concentration of the relevant biomarker at t = 0 in the group of individuals that showed severe fibrosis after 6 years follow up.

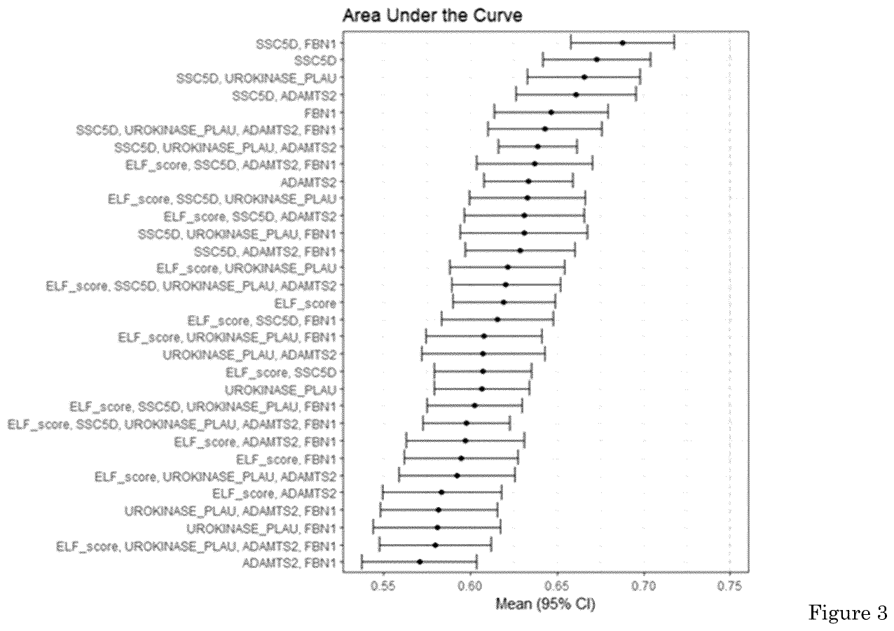

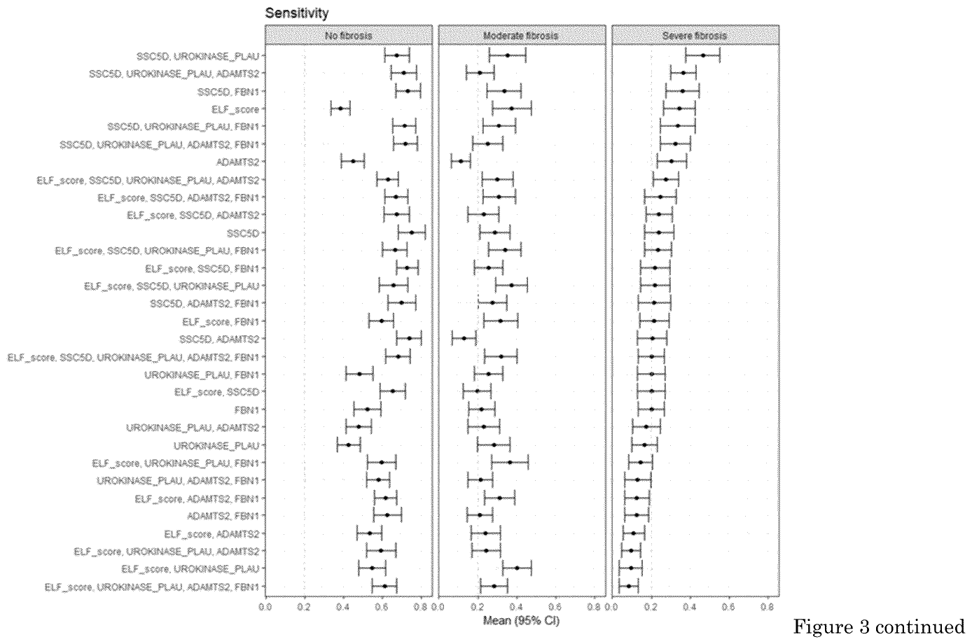

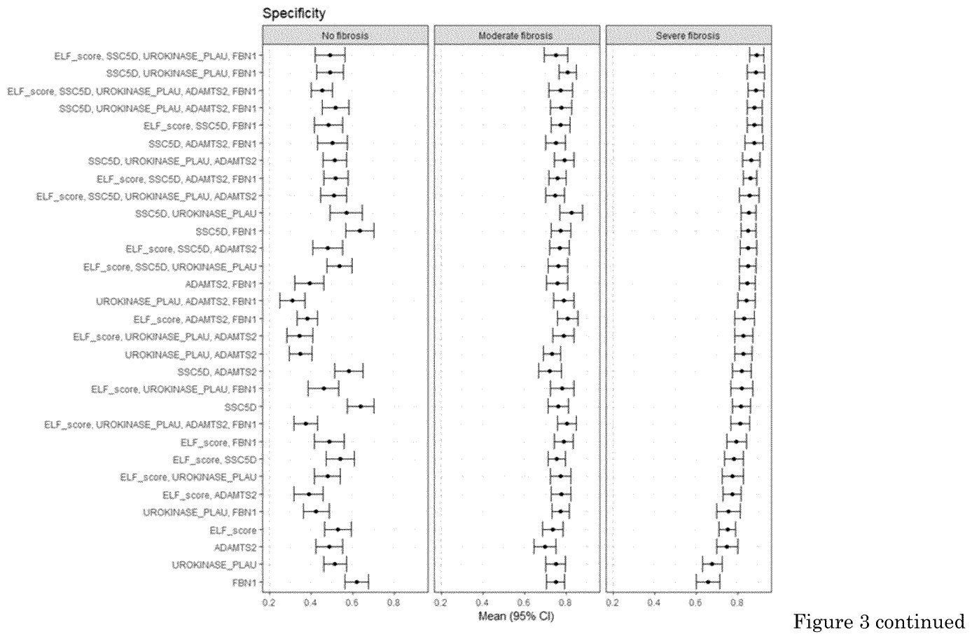

Next, using baseline serum data, Random Forest analysis showed that several biomarkers alone and in combination were able to predict the presence or absence of fibrosis after 6 years follow up (FIG. 3). The best prediction was observed using SSC5D alone resulting in predictive value of 68% (AUROC=0.68) using SSC5D with predictive value.

Predictive value (no fibrosis, moderate fibrosis and severe fibrosis) at 6 years follow up by ELF score was 62% (AUROC=0.62), which was improved by combination with our biomarkers.

1. A method for classifying a subject for being at risk of suffering from hepatic fibrosis or for being at risk of hepatic fibrosis progression, the method comprising determining the protein level of Scavenger Receptor Cysteine Rich Family Member With 5 Domains (SSC5D), Fibrillin 1 (FBN1), Thrombospondin 1 (THBS1), Urokinase-type plasminogen activator (uPA) and/or A disintegrin and metalloproteinase with thrombospondin motifs 2 (ADAMTS2) in a blood, serum or plasma sample of said subject and classifying the subject on the basis of said protein level.

2. A method for predicting a risk of development or progression of hepatic fibrosis in a subject, the method comprising determining the protein level of Scavenger Receptor Cysteine Rich Family Member With 5 Domains (SSC5D) Fibrillin 1 (FBN1), Thrombospondin 1 (THBS1), Urokinase-type plasminogen activator (uPA) and/or A disintegrin and metalloproteinase with thrombospondin motifs 2 (ADAMTS2) in a blood, serum or plasma sample of said subject and predicting risk of development or progression of hepatic fibrosis on the basis of said protein level.

3. The method according to any one of the preceding claims, comprising determining the protein level of SSC5D, FBN1 and/or THBS1 in said blood, serum or plasma sample and classifying the subject on the basis of the determined protein levels.

4. The method according to any one of the preceding claims, comprising determining the protein level of SSC5D in said blood, serum or plasma sample and classifying the subject on the basis of the determined protein levels.

5. The method according to any one of the preceding claims, comprising determining the protein level of at least two proteins selected from SSC5D, FBN1, THBS1, uPA and ADAMTS2 in said blood, serum or plasma sample and classifying the subject on the basis of the determined protein levels.

6. The method according to any one claims 1-5, comprising determining the protein levels of SSC5D and FBN1 in said blood, serum or plasma sample and classifying the subject on the basis of the determined protein levels.

7. The method according to any one of any one claims 1-5, comprising determining the protein levels of SSC5D, FBN1 and THBS1 in said blood, serum or plasma sample and classifying the subject on the basis of the determined protein levels.

8. The method according to any one of any one claims 1-5, comprising determining the protein levels of SSC5D and THBS1 in said blood, serum or plasma sample and classifying the subject on the basis of the determined protein levels.

9. The method according to any one of any one claims 1-5, comprising determining the protein levels of FBN1 and THBS1 in said blood, serum or plasma sample and classifying the subject on the basis of the determined protein levels.

10. The method according to any one of the preceding claims, further comprising prognosticating severity of hepatic fibrosis.

11. The method according to claim 10, comprising classifying a subject for being at risk of suffering from no, moderate or severe hepatic fibrosis.

12. A method for prognosticating severity of hepatic fibrosis in a subject, the method comprising determining the protein level of Scavenger Receptor Cysteine Rich Family Member With 5 Domains (SSC5D), Fibrillin 1 (FBN1), Thrombospondin 1 (THBS1), Urokinase-type plasminogen activator (uPA) and/or A disintegrin and metalloproteinase with thrombospondin motifs 2 (ADAMTS2) in a blood, serum or plasma sample of said subject and prognosticating severity of hepatic fibrosis on the basis of said protein level.

13. The method according to claim 12, comprising classifying the subject for being at risk of suffering from no, moderate or severe hepatic fibrosis.

14. The method according to claim 12 or 13, comprising determining the protein level of SSC5D, FBN1, ADAMTS2 and/or uPA in said blood, serum or plasma sample and classifying the subject on the basis of the determined protein levels.

15. The method according to claim 12 or 13, comprising determining the protein level of SSC5D in said blood, serum or plasma sample and classifying the subject on the basis of the determined protein levels.

16. The method according to any one of claims 12-15, comprising determining the protein level of at least two proteins selected from SSC5D, FBN1, THBS1, uPA and ADAMTS2 in said blood, serum or plasma sample and classifying the subject on the basis of the determined protein levels.

17. The method according to any one of claims 12-15, comprising determining the protein levels of SSC5D and uPA in said blood, serum or plasma sample and classifying the subject on the basis of the determined protein levels.

18. The method according to any one of claims 12-15, comprising determining the protein levels of SSC5D and ADAMTS2 in said blood, serum or plasma sample and classifying the subject on the basis of the determined protein levels.

19. The method according to any one of claims 12-15, comprising determining the protein levels of SSC5D, uPA, ADAMTS2 and FBN1 in said blood, serum or plasma sample and classifying the subject on the basis of the determined protein levels.

20. The method according to any one of claims 12-15, comprising determining the protein levels of SSC5D, ADAMTS2 and FBN1 in said blood, serum or plasma sample and classifying the subject on the basis of the determined protein levels.

21. The method according to any one of claims 12-15, comprising determining the protein levels of SSC5D, uPA and ADAMTS2 in said blood, serum or plasma sample and classifying the subject on the basis of the determined protein levels.

22. The method according to any one of claims 12-15, comprising determining the protein levels of SSC5D, uPA and FBN1 in said blood, serum or plasma sample and classifying the subject on the basis of the determined protein levels.

23. A method for analysing a blood, serum or plasma sample of a subject, the method comprising determining the protein level of Scavenger Receptor Cysteine Rich Family Member With 5 Domains (SSC5D), Fibrillin 1 (FBN1), Thrombospondin 1 (THBS1), Urokinase-type plasminogen activator (uPA) and/or A disintegrin and metalloproteinase with thrombospondin motifs 2 (ADAMTS2) in said sample.

24. A method for typing a blood, serum or plasma sample from subject, the method comprising determining the protein level of Scavenger Receptor Cysteine Rich Family Member With 5 Domains (SSC5D), Fibrillin 1 (FBN1), Thrombospondin 1 (THBS1), Urokinase-type plasminogen activator (uPA) and/or A disintegrin and metalloproteinase with thrombospondin motifs 2 (ADAMTS2) in a blood, serum or plasma sample of said subject and typing said sample on the basis of said protein level.

25. A method for classifying and treating a subject, the method comprising:
determining the protein level of Scavenger Receptor Cysteine Rich Family Member With 5 Domains (SSC5D), Fibrillin 1 (FBN1), Thrombospondin 1 (THBS1), Urokinase-type plasminogen activator (uPA) and/or A disintegrin and metalloproteinase with thrombospondin motifs 2 (ADAMTS2) in a blood, serum or plasma sample of said subject,
classifying the subject as being at risk of suffering from hepatic fibrosis, for being at risk of hepatic fibrosis progression or as suffering from progressive hepatic fibrosis based on said protein level,
providing treatment to the subject classified as being at risk of suffering from hepatic fibrosis.

26. The method according to claim 25 comprising comparing said determined protein level or a value for said protein level with a reference value, wherein said reference value is the protein level or levels of the same protein or proteins or a value for said protein level or levels in a blood, serum or plasma sample from one or more subjects not suffering from hepatic fibrosis or one or more subjects known to suffer from hepatic fibrosis.

27. The method according to any one of claims 23-26, comprising determining the protein level of at least two proteins selected from SSC5D, FBN1, THBS1, uPA and ADAMTS2 in said blood, serum or plasma sample.

28. A method for assigning subjects to a clinical trial for treatment or prevention of hepatic fibrosis or non-alcoholic steatohepatitis (NASH) associated with hepatic fibrosis, the method comprising classifying subjects as being at risk of suffering from hepatic fibrosis or for being at risk of hepatic fibrosis progression and/or prognosticating severity of hepatic fibrosis with a method according to any one of claims 1-22 or 26-27 and assigning subjects that are classified and/or prognosticated to said clinical trial.

29. The method according to any one of the preceding claims wherein the protein level or levels are compared with a reference or a value for the protein level or levels is compared with a reference value.

30. The method according to claim 29, wherein said reference value is the protein level or levels of the same protein or proteins in a blood, serum or plasma sample, or a value for said protein levels or levels, from subject or subjects not suffering from hepatic fibrosis, from a subject or subjects suffering from hepatic fibrosis, or from a subject or subjects suffering from moderate or severe hepatic fibrosis.

31. A kit of parts comprising means for determining protein level of Scavenger Receptor Cysteine Rich Family Member With 5 Domains (SSC5D) and one or more of Urokinase-type plasminogen activator (uPA), Fibrillin 1 (FBN1), A disintegrin and metalloproteinase with thrombospondin motifs 2 (ADAMTS2) and Thrombospondin 1 (THBS1), wherein said means comprises binding molecules specific for SSC5D and one or more of uPA, FBN1, ADAMTS2 and THBS1.
